(19) United States
(12) Patent Application Publication
(10) Pub. No.: US 2003/0144308 A1
(43) Pub. Date: Jul. 31, 2003

(54) Fructose 1,6-bisphosphatase inhibitors

(76) Inventors: Paul H. Bauer, (US); Stephen W. Wright, (US); Rodney C. Schnur, (US)

Correspondence Address: PFIZER INC., PATENT DEPARTMENT, MS8260-1611, EASTERN POINT ROAD, GROTON, CT, 06340, US

(21) Appl. No.: 10/251,073

(22) Filed: Sep. 20, 2002

Related U.S. Application Data
(60) Provisional application No. 60/324,751, filed on Sep. 24, 2001.

(51) Int. Cl. A61K 31/541; A61K 31/5377; A61K 31/517; C07D 417/14; C07D 413/14; C 07D 4 3/02

(52) U.S. Cl. 514/266.2; 544/284; 514/266.21; 514/266.23; 544/116; 544/60; 514/252.17; 514/228.2; 514/233.8

(57) ABSTRACT

The present invention relates to certain quinazoline compounds which have utility in the treatment of diabetes mellitus, hypercholesterolemia, hyperlipidemia, diabetic complications and cancer. The invention also relates to pharmaceutical compositions and kits comprising such quinazoline compounds and to methods of using such compounds in the treatment of diabetes mellitus, hypercholesterolemia, hyperlipidemia, diabetic complications and cancer.

### CROSS-REFERENCE TO RELATED APPLICATION

This application claims priority from U.S. provisional application No. 60/324,751, filed Sep. 24, 2001.

### FIELD OF THE INVENTION

The present invention relates to certain quinazoline compounds which have utility in the treatment of diabetes mellitus, hypercholesterolemia, hyperlipidemia, diabetic complications and cancer. The invention also relates to pharmaceutical compositions and kits comprising such quinazoline compounds and to methods of using such compounds in the treatment of diabetes mellitus, hypercholesterolemia, hyperlipidemia, diabetic complications and cancer.

### BACKGROUND OF THE INVENTION

Diabetes mellitus is characterized by metabolic defects in production and utilization of carbohydrates, resulting in elevated blood glucose or hyperglycemia due to the failure to maintain appropriate blood sugar levels. Research in the treatment of diabetes has centered on attempts to normalize fasting and postprandial blood glucose levels. Current treatments include administration of exogenous insulin, oral administration of drugs and dietary and/or exercise therapies.

Two major forms of diabetes mellitus are recognized. Type I diabetes, or insulin-dependent diabetes, is the result of an absolute deficiency of insulin, the hormone which regulates carbohydrate utilization. Type II diabetes, or non-insulin dependent diabetes, often occurs with normal, or even elevated levels of insulin and appears to be the result of the inability of tissues to respond appropriately to insulin. Complications of Type II diabetes include retinopathy, nephropathy, neuropathy, and coronary heart disease, and are believed to be triggered in part by excessive protein glycation, which in turn results from excessive levels of circulating glucose. Clinical studies have been carried out to define the primary defect that causes the elevated fasting blood glucose levels observed in Type II diabetics. The results have suggested that excessive hepatic glucose output is a principal cause (J. Clin. Invest. 1981, 68, 957; Diabetes 1988, 37, 667). Hepatic glucose output, in turn, derives from the breakdown of hepatic glycogen (glycogenolysis) and synthesis of glucose from precursors such as pyruvate (gluconeogenesis). Gluconeogenic flux has been shown to be excessive in Type 2 diabetics (Science 1991, 254, 573; . J. Clin. Invest 1992, 90, 1323; Diabetes, 1989, 38, 550; Diabetologia 1998, 41, 307). Gluconeogenesis is a highly regulated and well understood pathway.

Fructose-1,6-bisphosphatase is an enzyme found in the liver and kidney, and is a key enzyme in the regulation of hepatic gluconeogenesis. Liver fructose-1,6-bisphosphatase activity is elevated in insulin-deficient and insulin-resistant animal models (Endocrinol. Jpn. 1989, 36, 65; Biochem. J. 1970, 120, 95). The physiologically relevant form of fructose-1,6-bisphosphatase is a homotetramer (Ann. Rev. Physiol. 1992, 54, 885) which is subject to competitive substrate inhibition by fructose-2,6-bisphosphate (Proc. Natl. Acad. Sci. USA. 1981, 78, 2861; J. Biol Chem. 1981, 256, 3619) and to allosteric inhibition by adenosine 5′-monophosphate (AMP) (J. Biol. Chem. 1965, 240, 651). The regulation and molecular basis of fructose-1,6-bisphosphatase enzyme activity has been deduced from crystallographic studies utilizing the recombinant human fructose-1,6-bisphosphatase protein (J. Mol. Biol. 1994, 244, 609; J. Biol. Chem. 1994, 269, 27732). A reduction in hepatic glucose output will result from inhibition of fructose-1,6-bisphosphatase. A fructose-1,6-bisphosphatase inhibitor lowers blood glucose by inhibiting the elevated rate of gluconeogenesis present in diabetic patients.

Inhibition of fructose-1,6-bisphosphatase causes an increase in fructose-1,6-bisphosphate levels, which are useful in treating myocardial ischemia, tissue ischemia, coronary artery disease, atherosclerosis, asthma attacks, analgesic/sickle cell anemia and hypertension. Increased fructose-1,6-bisphosphate levels are also useful in cardiprotection, in improving cardiac function and in suppression of allograft/transplantation rejection. Further, increased fructose-1,6-bisphosphate levels are also useful in pre-diagnostic emergency treatment. Accordingly, the compounds of this invention have utility in the treatment of each of the above diseases or conditions.

Many of the current treatment regimes for cancer utilize compounds which inhibit DNA synthesis. Such compounds are toxic to cells generally but their toxic effect on the rapidly dividing tumor cells can be, beneficial. Alternative approaches to anti-cancer agents which act by mechanisms other than the direct inhibition of DNA synthesis have been explored in order to enhance the selectivity of action against cancer cells.

It is known that a cell may become cancerous by virtue of the transformation of a portion of its DNA into an oncogene (i.e. a gene which, on activation, leads to the formation of malignant tumor cells). Many oncogenes encode proteins which are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. It is known that such kinases are frequently aberrantly expressed in common human cancers such as breast cancer, gastrointestinal cancer such as colon, rectal or stomach cancer, leukemia, and ovarian, bronchial or pancreatic cancer. It has also been shown that epidermal growth factor receptor (EGFR) which possesses tyrosine kinase activity is mutated and/or overexpressed in many human cancers such as brain, lung, squamous cell, bladder, gastric, breast, head and neck, oesophageal, gynecological and thyroid tumors.

Accordingly, it has been recognized that inhibitors of receptor tyrosine kinases are useful as selective inhibitors of the growth of mammalian cancer cells. For example, erbstatin, a tyrosine kinase inhibitor selectively attenuates the growth in athymic nude mice of a transplanted human mammary carcinoma which expresses epidermal growth factor receptor tyrosine kinase (EGFR) but is without effect on the growth of another carcinoma which does not express the EGF receptor.

U.S. Pat. No. 5,736,534 discloses certain 4-heterocyclyl-substituted quinazoline derivatives for use as anti-cancer agents.

### SUMMARY OF THE INVENTION

The present invention is directed to compounds of Formula I,

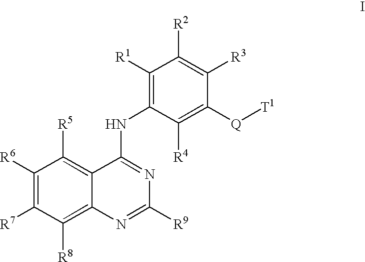

prodrugs thereof and pharmaceutically acceptable salts of said compounds and said prodrugs, wherein:

Q is pyrrolyl, pyrazolyl, oxazolyl, thiazolyl, isoxazolyl, isothiazolyl, oxadiazolyl, thiadiazolyl, triazolyl, furyl, quinolyl, imidazolyl, pyridyl or pyrimidyl;

T¹ is hydrogen, methyl, ethyl, OR¹⁰, SR¹⁰, cyano, cyclopropyl, cyclobutyl, NH₂, NHR¹⁰,

N(R¹⁰)₂, NHNH₂, CHR¹⁰OH, CH₂ORR¹⁰, COCH₃ or CON(R¹⁰)₂;

R¹, R², R³ and R⁴ are each independently hydrogen, halo, trifluoromethyl, (C₁-C₄)alkyl or (C₁-C₄)alkoxy;

R⁵ and R⁸ are each independently hydrogen, fluoro, chloro, hydroxy or methyl;

R⁶ and R⁷ are each independently (C₁-C₄)alkyl or (C₁-C₄)alkoxy;

R⁹ is hydrogen, cyclopropyl, cyclobutyl, (C₁-C₄)alkyl or (CH₂)ₘ—Y;

R¹⁰, for each occurrence, is independently hydrogen, methyl or ethyl;

m is 1, 2, 3 or 4;

Y is fluoro, chloro, bromo, hydroxy, N(R¹¹)₂, N-methylpiperazin-1-yl, thiazolidin-3-yl, thiomorpholin-4-yl, piperidin-1-yl, pyrrolidin-1-yl, morpholin-4-yl, imidazol-1-yl, (C₁-C₄)alkoxy, SR¹¹, SOR¹¹, SO₂R¹¹, CO₂H, CO₂(C₁-C₄)alkyl or CON(R¹¹)₂; and

R¹¹, for each occurrence, is independently hydrogen or (C₁-C₄)alkyl.

A preferred group of compounds of Formula I are those compounds, prodrugs thereof and pharmaceutically acceptable salts of said compounds and said prodrugs, wherein:

Q is oxazolyl, thiazolyl, isoxazolyl, pyridyl or pyrimidyl;

T¹ is hydrogen, methyl, OR¹⁰, CHR¹⁰OH or CON(R¹⁰)₂;

R¹, R², R³ and R⁴ are each independently hydrogen or fluoro, provided that only one of R¹, R², R³ or R⁴ is fluoro;

R⁵ and R⁸ are each hydrogen;

R⁶ is ethoxy;

R⁷ is ethoxy, n-propyl or isopropyl;

R⁹ is hydrogen or (CH₂)ₘ—Y;

Y is chloro, N(R¹¹)₂, piperidin-1-yl, pyrrolidin-1-yl, morpholin-4-yl, imidazol-1-yl, (C₁-C₄)alkoxy or SO₂R¹¹; and

R¹¹ is methyl.

This invention is also directed to pharmaceutical compositions comprising a therapeutically effective amount of a compound of Formula I. Preferably, such pharmaceutical compositions additionally comprise a pharmaceutically acceptable carrier, vehicle or diluent.

This invention is also directed to methods of treating diabetes, diabetic complications or cancer in a mammal comprising administering to said mammal a compound of Formula I. In a further embodiment, this invention is directed to methods of treating diabetes, diabetic complications, hypercholesterolemia, hyperlipidemia or cancer in a mammal comprising administering to said mammal a pharmaceutical composition as set forth in the immediately preceding paragraph. In one preferred embodiment of this invention, the methods treat Type I or Type II diabetes. In another preferred embodiment of this invention, the methods treat one or more diabetic complications, such as, but not limited to, diabetic neuropathy, diabetic nephropathy, diabetic cardiomyopathy or cataracts. In yet another preferred embodiment of this invention, the methods treat cancer.

In another aspect, the present invention provides pharmaceutical compositions comprising a compound of Formula I, a prodrug thereof, or a pharmaceutically acceptable salt of the compound or prodrug.

In a preferred embodiment of the methods of treating diabetes, the diabetes is Type I diabetes.

In another preferred embodiment of the methods of treating diabetes, the diabetes is Type II diabetes.

Also provided are methods of treating hypercholesterolemia, which methods comprise administering to patients having, or at risk of having, hypercholesterolemia, a therapeutically effective amount of a compound of Formula I, a prodrug thereof, or a pharmaceutically acceptable salt of the compound or prodrug.

Also provided are methods of treating hyperlipidemia, which methods comprise administering to patients having, or at risk of having, hyperlipidemia, a therapeutically effective amount of a compound of Formula I, a prodrug thereof, or a pharmaceutically acceptable salt of the compound or prodrug.

Also provided are methods of treating cancer, which methods comprise administering to patients having, or at risk of having, cancer, a therapeutically effective amount of a compound of Formula I, a prodrug thereof, or a pharmaceutically acceptable salt of the compound or prodrug.

Certain of the compounds of Formula I, for example those which have free carboxylic acid functionality, form pharmaceutically-acceptable cation salts by reacting the free acid forms with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate), employing a solvent (e.g., ethyl acetate) from which the desired cationic salt precipitates, or can be otherwise isolated by concentration and/or addition of a non-solvent.

The acid addition salts of the compounds of the present invention are readily prepared by reacting the base forms with the appropriate acid. When the salt is of a monobasic acid (e.g., the hydrochloride, the hydrobromide, the p-toluenesulfonate or the acetate), the hydrogen form of a dibasic acid (e.g., the hydrogen sulfate or the succinate) or the dihydrogen form of a tribasic acid (e.g., the dihydrogen phosphate or the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

The term “prodrug” means a compound that is transformed in vivo to yield a compound of Formula I or a pharmaceutically acceptable salt of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in “Bioreversible Carriers in Drug Design,” ed. Edward B. Roche, American Pharmaceutical Association and Pergamon Press, 1987.

For example, if a compound of Formula I contains a carboxylic acid functional group, an ester prodrug may be formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

The amino acid prodrugs of this invention are readily prepared by conventional peptide coupling reactions coupling a free amino or carboxylic group of the compound of Formula I with an amino acid or a polypeptide, e.g. dipeptide, chain. The coupling reaction is generally conducted at a temperature of about −30° C. to about 80° C., preferably about 0° C. to about 25° C. Suitable coupling reagents are usually present, such as dicyclohexylcarbodiimide with hydroxybenzotriazole (HBT), N-3-dimethylaminopropyl-N′-ethylcarbodiimide with HBT, 2-ethoxy-1-ethoxycarbonyl-1,2-dihydroquinoline, carbonyl diimidazole with HBT, or diethylphosphoryl-cyanide. The reaction is generally conducted in an inert solvent such as acetonitrile, methylene chloride, chloroform, dimethylformamide, dioxane, tetrahydrofuran, dimethoxyethane, or water, or a mixture of two or more such solvents.

Similarly, if a compound of Formula I contains an alcohol functional group, a prodrug may be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

Further, if a compound of Formula I contains an amine functional group, a prodrug may be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural (α-aminoacyl-natural α-aminoacyl, —C(OH)C(O)OY wherein Y is H, (C₁-C₆)alkyl or benzyl, —C(OY₀)Y₁ wherein Y₀ is (C₁-C₄) alkyl and Y₁ is ((C₁-C₆)alkyl, carboxy(C₁-C₆)alkyl, amino(C₁-C₄)alkyl or mono-N- or di-N,N—(C₁-C₆)alkylaminoalkyl, —C(Y₂)Y₃ wherein Y₂ is H or methyl and Y₃ is mono-N- or di-N,N—(C₁-C₆)alkylamino, morpholino, piperidin-1-yl or pyrrolidin-1-yl.

Ester, carbonate or carbamate prodrugs of this invention are readily prepared by reaction of a free hydroxyl or amino group of the compound of Formula I with an activated carbonyl containing molecule such as acetyl chloride or ethyl chloroformate. The reaction can be carried out neat or in the presence of a reaction inert solvent such as methylene chloride, at a temperature from about −78° C. to about 100° C. Alcohols can also be reacted with cyanogen chloride in the presence of a Lewis acid to form carbamates.

The compounds of Formula I may contain asymmetric or chiral centers, and, therefore, exist in different stereoisomeric forms. It is intended that all stereoisomeric forms of the compounds of Formula I, e.g., enantiomers, diastereomers and mixtures thereof, including racemic mixtures, form part of the present invention. In addition, the present invention embraces all geometric and positional isomers. For example, if a compound of Formula I contains a double bond, both the cis- and trans- forms, as well as mixtures, are embraced within the scope of the invention.

Diasteromeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Other methods of resolving enantiomers from racemic mixtures are also well known, e.g., chromatographic separation using a chiral support. Also, some of the compounds of Formula I may be atropisomers (e.g., substituted biaryls) and are considered as part of this invention. The present invention also embraces all polymorphic forms of the compounds, prodrugs and salts of Formula I.

The compounds, prodrugs and salts of Formula I may exist in unsolvated as well as solvated forms with pharmaceutically acceptable solvents such as water, ethanol, and the like, and it is intended that the invention embrace both solvated and unsolvated forms.

It is also possible that the compounds of Formula I may exist in different tautomeric forms, and all such forms are embraced within the scope of the invention, even though a compound may only be named herein as one specific tautomer. For example, all of the tautomeric forms of the imidazole moiety are included in the invention. Also, for example, all keto-enol or imine-enamine forms of the compounds are included in the invention.

It is also intended that the invention disclosed herein encompass compounds of Formula I that may be synthesized in vitro using laboratory techniques, such as those well known to the synthetic organic chemist of ordinary skill, or~synthesized using in vivo techniques, such as through metabolism, fermentation, digestion, and the like. It is also intended that the compounds of Formula I may be synthesized using a combination of in vitro and in vivo techniques.

The present invention also embraces isotopically-labelled compounds, prodrugs and salts of Formula I, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. The compounds of Formula I, the stereoisomers and prodrugs thereof, and the pharmaceutically acceptable salts of the compounds, stereoisomers, or prodrugs which contain the aforementioned isotopes and/or other isotopes of other atoms are intended to be within the scope of this invention.

Certain isotopically-labelled compounds, prodrugs and salts of Formula I, for example those into which radioactive isotopes such as ³H and ¹⁴C are incorporated, are useful in compound and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labelled compounds of Formula I can generally be prepared by carrying out the procedures analogous to those disclosed in the Schemes and/or in the Examples below, by substituting a readily available isotopically labelled reagent for a non-isotopically labelled reagent.

The term “halo”, as used herein, unless otherwise indicated, is interchangeable with the term “halogen,” and includes chloro, fluoro, bromo and iodo.

The expression “reaction inert solvent” refers to any solvent or mixture of solvents which does not interact with starting materials, reagents, intermediates or products in a manner which adversely affects the reaction or yield of the desired product.

The term “alkyl” denotes a straight or branched chain hydrocarbon. Representative examples of alkyl groups comprise methyl, ethyl, propyl, isopropyl, butyl, isobutyl, tert-butyl, sec-butyl, pentyl, and hexyl. Generally preferred alkyl groups are (C₁-C₄)alkyl.

The term “alkoxy” denotes an alkyl group bonded to an oxygen atom. Representative examples of alkoxy groups include methoxy, ethoxy, tert-butoxy, propoxy, and isobutoxy. Preferred alkoxy groups are (C₁-C₄)alkoxy.

The term “alkenyl” denotes a branched or straight chain hydrocarbon having one or more carbon-carbon double bonds.

The term “alkynyl” denotes a branched or straight chain hydrocarbon having one or more carbon-carbon triple bonds.

The term “cycloalkyl” denotes a cyclic hydrocarbon. Examples of cycloalkyl groups include cyclopropyl, cyclobutyl, cyclopentyl, cyclohexyl and cycloheptyl. The cycloalkyl groups may be substituted or unsubsituted with from one to four substitutents.

The term “perfluoroalkyl” denotes an alkyl group in which all of the hydrogen atoms have been replaced with fluorine atoms.

The term “acyl” denotes a group derived from an organic acid (—COOH) by removal of the hydroxy group (—OH).

The term “aryl” denotes a cyclic, aromatic hydrocarbon. Examples of aryl groups include phenyl, naphthyl and biphenyl. The aryl group can be unsubstituted or substituted.

The term “heteroatom” includes oxygen, nitrogen, sulfur, and phosphorous.

The term “heteroaryl” denotes a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, oxazolyl, thiazolyl, isoxazolyl, pyrazolyl, isothiazolyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, 1,3,5-triazinyl, 1,2,4-triazinyl, 1,2,3-triazinyl, naphthyridinyl, quinoxalinyl, 1,2,3-oxadiazolyl, 1,2,4-oxadiazolyl, 1,2,5-oxadiazolyl, 1,3,4-oxadiazolyl, 1,2,3-triazolyl, 1,2,4-trizaolyl, 1,3,4-thiadiazolyl and benzo[b]thienyl. Preferred heteroaryl groups are five and six membered rings and contain from one to three heteroatoms independently selected from O, N, and S. The heteroaryl group, including each heteroatom, can be unsubstituted or substituted with from 1 to 4 substituents, as chemically feasible. For example, the heteroatom S may be substituted with one or two oxo groups, which may be shown as ═O.

The term “heterocycloalkyl” denotes a cycloalkyl group in which one or more of the carbon atoms has been replaced with heteroatoms. If the heterocycloalkyl group contains more than one heteroatom, the heteroatoms may be the same or different. Examples of heterocycloalkyl groups include tetrahydrofuryl, morpholinyl, piperazinyl, piperidyl, and pyrrolidinyl. Preferred heterocycloalkyl groups are five and six membered rings and contain from one to three heteroatoms independently selected from O, N, and S. A heterocycloalkyl group, including each heteroatom, can be unsubstituted or substituted with from 1 to 4 substituents, as chemically feasible. For example, the heteroatom S may be substituted with one or two oxo groups, which may be shown as ═O.

Further examples of rings are cyclopentyl, cyclohexyl, cycloheptyl, cyclooctyl and phenyl. Further exemplary five membered rings are pyrrolyl, 2-pyrrolinyl, 3-pyrrolinyl, pyrrolidinyl, 1,3-dioxolanyl, 2-imidazolinyl, imidazolidinyl, 2-pyrazolinyl, pyrazolidinyl, 1,2-dithiolyl, 1,3-dithiolyl, 3H-1,2-oxathiolyl, 3H-1,2,3-dioxazolyl, 1,2,4-dioxazolyl, 1,3,2-dioxazolyl, 1,3,4-dioxazolyl, 5H-1,2,5-oxathiazolyl and 1,3-oxathiolyl.

Further exemplary six membered rings are 2H-pyranyl, 4H-pyranyl, piperidinyl, 1,2-dioxinyl, 1,3-dioxinyl, 1,4-dioxanyl, morpholinyl, 1,4-dithianyl, piperazinyl, thiomorpholinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl and 1,4,2-oxadiazinyl.

A cyclic ring group may be bonded to another group in more than one way. If no particular bonding arrangement is specified, then all possible arrangements are intended. For example, the term “pyridyl” includes 2-, 3-, or 4-pyridyl, and the term “thienyl” includes 2-, or 3-thienyl.

The term “substituted” means that a hydrogen atom on a molecule has been replaced with a different atom or molecule. The atom or molecule replacing the hydrogen atom is called a substituent.

The symbol “—” represents a covalent bond.

The term “radical” denotes a group of atoms that behaves as a single atom in a chemical reaction, e.g., an organic radical is a group of atoms which confers characteristic properties on a compound containing it, or which remains unchanged during a series of reactions.

The phrase “therapeutically effective amount” means an amount of a compound of Formula I, a prodrug thereof, or a pharmaceutically acceptable salt of the compound or prodrug, or a combination of a compound of Formula I, a prodrug thereof, or a pharmaceutically acceptable salt of the compound or prodrug, and another compound to be described in detail hereinbelow, which amount ameliorates, attenuates, or eliminates one or more diseases or conditions, or prevents or delays the onset of one of more diseases or conditions.

The term “mammal” means animals such as dogs, cats, cows, horses, sheep, and humans. Preferred mammals are humans, including both male and female sexes.

The phrase “pharmaceutically acceptable” indicates that the substance or composition must be compatible chemically and/or toxicologically, with other ingredients comprising a formulation, and/or the patient being treated therewith.

The terms “treating”, “treat”, or “treatment” embrace both preventative, i.e., prophylactic, and palliative treatment.

The term “cancer” includes, but is not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

### DETAILED DESCRIPTION OF THE INVENTION

A process for the preparation of a compound of Formula I as defined above is provided as a feature of the invention and is illustrated by the following procedures in which the meanings of generic radicals are as given above unless otherwise qualified. The process can be effected, generally, by reacting a compound of formula II:

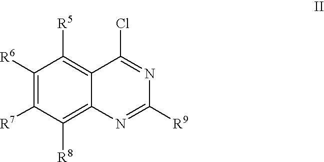

wherein R⁵, R⁶, R⁷, R⁸ and R⁹ are each as defined above, with a compound of formula III:

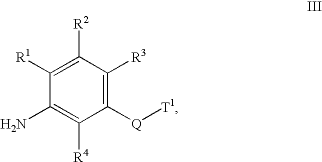

wherein R¹, R², R³, R⁴, Q and T¹ are each as defined above.

The reaction is typically implemented by stirring in a polar solvent: a lower alcohol such as methanol, ethanol or isopropanol; an ether such as diethyl ether, tetrahydrofuran or dioxane; a chlorinated hydrocarbon such as methylene chloride, chloroform or carbon tetrachloride; an aromatic hydrocarbon such as toluene, benzene or xylene; DMF, DMSO, or a mixture of any of these solvents. Ethanol and isopropanol are particularly suitable. The reaction is typically carried out at a temperature of about 20° C. to about 130° C., optionally in the presence of a base or acid to help effect the reaction. Suitable bases include include lower trialkylamines, sodium carbonate, potassium carbonate, sodium bicarbonate, potassium bicarbonate, pyridine, N,N-dimethylaniline, 2,6-lutidine and 2,4,6-collidine, 2,6-Lutidine is particularly suitable. Suitable acids include hydrochloric acid, sulfuric acid, trifluoroacetic acid, methanesulfonic acid and toluenesulfonic acid. Hydrochloric acid is particularly suitable. The preparation of intermediate compounds II and III is described below.

Compounds of Formula I may also be prepared, generally, by reacting a compound of formula X

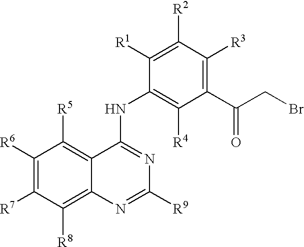

with a compound of the formula T¹SNH₂. Many compounds of the formula T¹CSNH₂ are known in the literature, or they may be prepared by reacting a compound of the formula T¹N with thioacetamide as described by Taylor (J. Am. Chem. Soc., 1960, 82, 2656) or, alternatively, by reacting a compound of the formula T¹ONH₂ with phosphorus pentasulfide or 2,4-bis(4-methoxyphenyl)-1,3-dithia-2,4-diphosphetane-2,4-disulfide (“Lawesson's Reagent”) as described by Cava (Tetrahedron, 1985, 41, 5061). The reaction is typically implemented by stirring the compound of formula X in a polar solvent: a lower alcohol such as methanol, ethanol or isopropanol; an ether such as diethyl ether, tetrahydrofuran or dioxane; a chlorinated hydrocarbon such as methylene chloride, chloroform or carbon tetrachloride; an aromatic hydrocarbon such as benzene, toluene or xylene; DMF; DMSO; or a mixture of such solvents. Ethanol and isopropanol are particularly suitable. The reaction is typically carried out at a temperature of about 20° C. to about 130° C., optionally in the presence of a base to facilitate the reaction. Suitable bases include include lower trialkylamines, sodium carbonate, potassium carbonate, sodium bicarbonate and potassium bicarbonate.

Compounds of Formula I may also be prepared, generally, by reacting a compound of Formula I in which R⁹ is (CH₂)ₘCl or (CH₂)ₘBr with a primary or secondary amine of formula HN(R¹¹)₂, or a heterocyclic amine such as piperidine, pyrrolidine, morpholine, imidazole, thiazolidine, or thiomorpholine.

This reaction is typically implemented by stirring in a polar solvent such as a lower alcohol such as methanol, ethanol or isopropanol; an ether such as diethyl ether, tetrahydrofuran or dioxane; an aromatic hydrocarbon such as benzene, toluene or xylene; DMF; DMSO; or a mixture of these solvents. Ethanol, 1,4-dioxane, tetrahydrofuran and isopropanol are particularly suitable. The reaction is typically carried out at a temperature of about 0° C. to about 130° C., optionally in the presence of a base to help effect the reaction. Suitable bases include lower trialkylamines, sodium carbonate, potassium carbonate, sodium bicarbonate or potassium bicarbonate.

Many compounds of Formula II are known in the literature, and may be prepared by published procedures or by methods well known to one skilled in the art, for example by reacting a compound of formula IV with a chlorinating agent, as described by Brown (Comprehensive Heterocyclic Chemistry, Volume 3, 1984 (Pergamon Press, Oxford, UK) pages 89-90 and references cited therein):

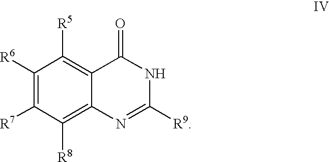

Suitable chlorinating agents for this reaction include phosphorus oxychloride, phosphorus pentachloride, thionyl chloride, oxalyl chloride, triphenylphosphine dichloride and phosgene. Phosphorus oxychloride is particularly suitable. The reaction may be carried out neat or in the presence of a reaction inert solvent. Suitable solvents include chlorinated hydrocarbons such as methylene chloride, chloroform and carbon tetrachloride; aromatic hydrocarbons such as benzene, toluene and xylene; aliphatic hydrocarbons such as hexanes; or a mixture of such solvents. The reaction may be carried out in the presence or absence of a chloride salt. Suitable chloride salts include, but are not limited to, a tertiary amine hydrochloride, a quaternary ammonium chloride and lithium chloride. Triethylamine hydrochloride is particularly suitable.

Many compounds of formula IV are known in the literature, or may be prepared by published procedures or by methods well known to one skilled in the art, for example by treating a compound of formula VI with a nitrile of formula R⁹CN or an amide of formula R⁹CONH₂ (“Niementowski synthesis”) as described by Brown (Comprehensive Heterocyclic Chemistry, Volume 3, 1984 (Pergamon Press, Oxford, UK) pages 107-116 and references cited therein):

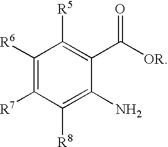

Many compounds of formula III are known in the literature, and may be prepared by published procedures or by methods well known to one skilled in the art, for example by reducing a compound of formula V, as described by Larock (Comprehensive Organic Transformations, 1989 (VCH Publishers, New York, N.Y.) pages 411-417 and references cited therein):

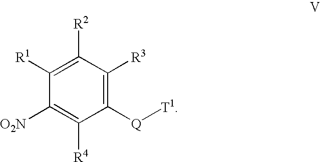

Suitable reducing agents include tin(II) chloride, tin and hydrochloric acid, iron and acetic acid, iron and hydrochloric acid, sodium hydrosulfite, sodium sulfide, hydrogen gas with a metal catalyst, and formic acid or a formate salt with a metal catalyst. It is preferred to carry out this reduction with tin(II) chloride or hydrogen gas in the presence of a metal catalyst. Suitable metal catalysts include palladium or Raney® nickel (Aldrich Chemical Co., 1001 West Saint Paul Avenue, Milwaukee, Wis. 53233), optionally on an inert solid support such as activated carbon.

Many compounds of formula V are known in the literature, or may be prepared by published procedures or by methods well known to one skilled in the art. For example, when Q is thiazolyl, compounds of formula V may be prepared by the Hantzsch thiazole synthesis by treating a compound of formula VII with a compound of the formula T¹SNH₂ as described by Gilchrist (Heterocyclic Chemistry, 1985 (John Wiley & Sons; New York, N.Y.) page 209):

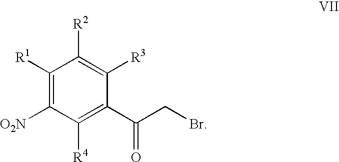

When Q is thiazolyl, the compound of formula V may alternatively be prepared by treating a compound of formula Vil with a compound of the formula T¹COCH₂Br, as described in the Gilchrist reference above.

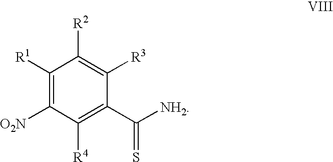

When Q is pyridyl the compound of formula V may be prepared by treating a compound of formula IX with nitric acid, as described by Katritzky (J. Chem. Soc.; 1960; 1511).

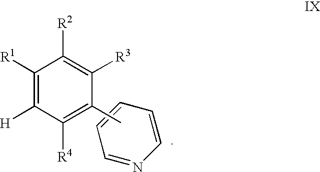

Starting materials for the above procedures are either commercially available or may be prepared by procedures well known in the art of organic chemistry, by techniques analogous to the synthesis of known compounds or by techniques analogous to the above described procedures or to the procedures described in the examples.

Conventional methods and/or techniques of purification and separation known to those skilled in the art can be used to isolate the compounds of this invention. Such techniques include all types of chromatography (HPLC, column chromatography using common adsorbents such as silica gel or alumina gel, and thin layer chromatography), recrystallization, and differential (i.e., liquid-liquid) extraction techniques.

The compounds of Formula I of the instant invention inhibit the enzyme fructose-1,6-bisphosphatase and as such have utility in the treatment of diabetes. The compounds of Formula I of the instant invention inhibit the endothelial growth factor receptor tyrosine kinase and as such have utility in the treatment of cancer. The utility of the compounds of Formula I of the present invention as medical agents in the treatment of diseases, such as are detailed herein in mammals (e.g., humans) for example, diabetes and cancer, is demonstrated by the activity of the compounds of Formula I of this invention in conventional assays. Such assays also provide a means whereby the activities of the compounds of Formula I of this invention can be compared with the activities of other known compounds. The results of these comparisons are useful for determining dosage levels in mammals, including humans, for the treatment of such diseases. Such assays are set forth below.

The compounds of this invention are potent inhibitors of the erbB family of oncogenic and protooncogenic protein tyrosine kinases such as epidermal growth factor receptor (EGFR), erbB2, HER3, or HER4 and thus are all adapted to therapeutic use as antiproliferative agents (e.g., anticancer) in mammals, particularly humans. In particular, the compounds of this invention are therapeutants or prophylactics for the treatment of a variety of benign or malignant human tumors (renal, liver, kidney, bladder, breast, gastric, ovarian, colorectal, prostate, pancreatic, lung, vulval, thyroid, hepatic carcinomas, sarcomas, glioblastomas, various head and neck tumors), and other noncancerous hyperplastic disorders such as benign hyperplasia of the skin (e.g., psoriasis) or prostate (e.g., BPH). Such activity against benign disorders can be determined by standard assays such as described in J. Invest. Dermatol. 98, 296-301 (1992). It is in addition expected that compounds of the present invention possess activity against a range of leukemias and lymphoid malignancies.

The compounds of Formula I also potentiate responses to conventional cancer chemotherapies and radiotherapy in a dose and schedule-dependent manner based upon the substantial synergy observed between neutralizing anti-EGFR antibodies and conventional chemotherapeutants (J. Nat. Cancer Inst. 85, 1327-1333 (1993); and Cancer. Res. 53, 4637-4642 (1993)).

The compounds of Formula I are also useful in the treatment of additional disorders in which aberrant expression, ligand/receptor interactions, activation, or signalling events related to various protein tyrosine kinases, whose activity is inhibited by the agents of Formula I, are involved.

Such disorders include those of neuronal, glial, astrocytal, hypothalamic, and other glandular, macrophagal, epithelial, stromal, and blastocoelic nature in which aberrant function, expression, activation or signaling of the erbB tyrosine kinases may be involved. Compounds of Formula I also have therapeutic utility in inflammatory, angiogenic and immunologic disorders involving both identified and as yet unidentified tyrosine kinases which are inhibited by compounds of Formula I.

Fructose-1,6-bisphosphatase Enzyme Assay: Fructose-1,6-bisphosphatase (F16BP) is assayed by measuring its ability to hydrolyze inorganic phosphate from fructose-1,6-bisphosphate using a modification of a reported method (Biochem. J. 1994, 298, 395). Specifically, 60 ng of recombinant human F16BPase (produced in a manner similar to J. Biol. Chem. 1993, 268, 9466) in 20 μL is added to 80 μL of substrate and a compound in a 96-well microtiter plate to start the reaction. The final solution consists of 50 mM HEPES (catalog #H7523, Sigma Chemical Company, PO Box 14508, St. Louis, Mo. 63178) pH=7.2, 100 mM KCl (Sigma catalog #P3911), 2 mM MgCI₂ (Sigma catalog #M9272), 2 mM EGTA (Sigma catalog #E0396), 1 mM DTT (Sigma atalog #D5545), and 500 1 μM fructose-1,6-bisphosphate (Sigma catalog #752-1). The solution is incubated for 40 minutes at room temperature without preincubation of compound with enzyme. The phosphate released by the enzyme is measured spectrophotometrically using a Titertek Multiscann MCC 340 (Titertek, 330 Wynn Drive, Huntsville, Ala. 35805) at 620 nm, after allowing the phosphate to form a complex for 10 minutes after the addition of 150 μL of ammonium molybdate/malachite green (AM/MG) solution. To prepare the AM/MG solution, one volume of 4.2% ammonium molybdate (w/v, Sigma catalog #A7302) in 4 molar aqueous hydrochloric acid is added to three volumes of 0.045% malachite green (w/v, Sigma catalog #M9636) and 0.01% Tween®-20 (v/v, Sigma catalog #P1379) in water. The AM/MG solution is stirred at room temperature for 30 minutes and filtered through a 0.22 micron filter before being added to each reaction well. Under these conditions, the assay is linear with time and enzyme concentration, and it is possible to detect allosteric inhibitors of F16BP (IC₅₀ of AMP against the human enzyme=0.8 μM).

Tyrosine kinase Enzyme Assay: The in vitro activity of these compounds in inhibiting the receptor tyrosine kinase (and thus subsequent proliferative response, e.g., cancer) may be determined by a procedure as detailed below.

Activity of compunds of Formula I in vitro can be determined by the amount of inhibition of the phosphorylation of an exogenous substrate (e.g., Lys₃- gastrin or polyGluTyr (4:1) random copolymer (J. Biol. Chem. 267 (29), 20638-47 (1992)) on tyrosine by epidermal growth factor receptor kinase by a test compound relative to a control. Affinity purified, soluble human EGF receptor (96 ng) is obtained according to the procedure in Methods in Enzymology 146, 82-88 (1987) from A431 cells (American Type Culture Collection, Rockville, Md.) and preincubated in a microfuge tube with EGF (2 μg/ml) in phosphorylation buffer with vanadate (PBV: 50 mM HEPES, pH 7.4; 125 mM NaCl; 24 mM MgCl₂; 100 μM sodium orthovanadate), in a total volume of 10 μl, for 20-30 minutes at room temperature. The test compound, dissolved in DMSO, is diluted in PBV, and 10 pl is mixed with the EGF receptor /EGF mix, and incubated for 10-30 minutes at 30° C. The phosphorylation reaction is initiated by addition of 20 μl ³³P-ATP/ substrate mix (120 μM Lys₃-Gastrin, 50 mM Hepes pH 7.4, 40 pM ATP, 2 μCi γ-[³³P]-ATP) to the EGFr/EGF mix and incubated for 20 minutes at room temperature. The reaction is stopped by addition of 10 μl stop solution (0.5 M EDTA, pH 8; 2 mM ATP) and 6 μl 2N HCl. The tubes are centrifuged at 14,000 RPM, 4° C., for 10 minutes. Thirty-five microliters of supernatant from each tube is pipetted onto a 2.5 cm circle of Whatman P81 paper, bulk washed four times in 5% acetic acid, 1 liter per wash, and then air dried. This results in the binding of substrate to the paper with loss of free ATP on washing. The [³³P] incorporated is measured by liquid scintillation counting. Incorporation in the absence of substrate (e.g., Iys₃-gastrin) is subtracted from all values as a background and percent inhibition is calculated relative to controls without test compound present.

Activity of compounds of Formula I in vivo can be determined by the amount of inhibition of tumor growth by a test compound relative to a control. The tumor growth inhibitory effects of various compounds are measured according to the methods of Cancer Res., 35, 2434-2439 (1975) and Cancer Chemother. Rep. (Part 2)”, 5, 169-186 (1975), with slight modifications. Tumors are induced in the left flank by s.c. injection of 1×10⁶ log phase cultured tumor cells (human MDA-MB-468 breast or human HN5 head and neck carcinoma cells) suspended in 0.10 ml RPMI 1640. After sufficient time has elapsed for the tumors to become palpable (2-3 mm in diameter) the test animals (athymic mice) are treated with compound (formulated by dissolution in DMSO typically at a concentration of 50 to 100 mg/mL followed by 1:9 dilution into 0.1% Pluronice P105 (BASF, 3000 Continental Drive-North, Mount Olive, N.J. 07828) in 0.9% saline) by the intraperitoneal (ip) or oral (po) routes of administration twice daily (i.e., every 12 hours) for 5 consecutive days. In order to determine a tumor growth inhibition, the tumor is measured in millimeters with Vernier calipers across two diameters and the tumor size (mg) is calculated using the formula: Tumor weight (TuW)=(length×[width]²)/2, according to the methods of Geran, R.I., et al. “Protocols for Screening Chemical Agents and Natural Products Against Animal Tumors and Other Biological Systems”, Third Edition, Cancer Chemother. Rep., 3, 1-104 (1972). Results are expressed as percent inhibition, according to the formula: Inhibition (%)=(TuW_{control}−TuWₜₑₛₜ)/TuW_{control}×100%. The flank site of tumor implantation provides reproducible dose/response effects for a variety of chemotherapeutic agents, and the method of measurement (tumor diameter) is a reliable method for assessing tumor growth rates.

In one aspect, the present invention concerns the treatment of diabetes, including impaired glucose tolerance, insulin resistance, insulin dependent diabetes mellitus (Type I), and non-insulin dependent diabetes mellitus (NIDDM or Type II). Also included in the treatment of diabetes are diabetic complications related thereto, including neuropathy, nephropathy, retinopathy, cataracts and the like.

The preferred type of diabetes to be treated by the compounds of Formula I, prodrugs thereof and the pharmaceutically acceptable salts of the compounds or prodrugs, is non-insulin dependent diabetes mellitus, i.e. NIDDM or Type II.

Diabetes can be treated by administering to a patient having diabetes (Type I or Type II), insulin resistance, impaired glucose tolerance, or any of the diabetic complications such as neuropathy, nephropathy, retinopathy or cataracts, a therapeutically effective amount of a compound of Formula I, a prodrug thereof, or a pharmaceutically acceptable salt of the compound or prodrug. It is also intended that diabetic patients can be treated by administering a compound of Formula I, a prodrug thereof, or a pharmaceutically acceptable salt of the compound or prodrug together with one or more anti-diabetic agents.

Representative agents that can be used to treat diabetes in combination with the compounds of Formula I, the prodrugs thereof, and the pharmaceutically acceptable salts of the compounds and prodrugs, include insulin and insulin analogs (e.g. LysPro insulin); inhaled insulin; GLP-1 (7-37) (insulinotropin) and GLP-1 (7-36)-NH₂; sulfonylureas and analogs: chlorpropamide, glibenclamide, tolbutamide, tolazamide, acetohexamide, glypizide, glimepiride, repaglinide, meglitinide; biguanides: metformin, phenformin, buformin; α2-antagonists and imidazolines: midaglizole, isaglidole, deriglidole, idazoxan, efaroxan, fluparoxan; other insulin secretagogues: linogliride, A-4166; glitazones: ciglitazone, pioglitazone, rosiglitazone, englitazone, troglitazone, darglitazone, BRL49653; fatty acid oxidation inhibitors: clomoxir, etomoxir; α-glucosidase inhibitors: acarbose, miglitol, emiglitate, voglibose, MDL-25,637, camiglibose, MDL-73,945; β-agonists: BRL 35135, BRL 37344, RO 16-8714, ICI D7114, CL 316,243; phosphodiesterase inhibitors: L-386,398; lipid-lowering agents: benfluorex; antiobesity agents: fenfluramine; vanadate and vanadium complexes (e.g. Naglivan®) and peroxovanadium complexes; amylin antagonists; glucagon antagonists; gluconeogenesis inhibitors; somatostatin analogs; antilipolytic agents: nicotinic acid, acipimox and WAG 994. Also intended to be used in combination with the compounds of Formula I, the prodrugs thereof, and the pharmaceutically acceptable salts of the compounds and prodrugs, are pramlintide (Symlin™), AC 2993 and nateglinide. Any agent, or combination of agents, can be administered as described hereinabove.

In addition, the compounds of Formula I, the prodrugs thereof, and the pharmaceutically acceptable salts of the compounds and prodrugs, can be used in combination with one or more aldose reductase inhibitors such as but not limited to zopolrestat, zenarestat, epalrestat, fidarestat, ponalrestat and tolrestat; glycogen phosphorylase inhibitors such as ingliforib; sorbitol dehydrogenase inhibitors, NHE-1 inhibitors and/or glucocorticoid receptor antagonists.

Other glycogen phosphorylase inhibitors which are useful in combination with the compounds, prodrugs and salts of Formula I will be known to those skilled in the art (e.g., International Application Publication No. WO 95/24391 and those inhibitors disclosed in U.S. Pat. No. 5,952,363). The following publications also disclose glycogen phosphorylase inhibitors that can be used in accordance with methods of the present invention: U.S. Pat. No. 6043091; U.S. Pat. No. 4,786,641 (BayR³⁴⁰¹); U.S. Pat. No. 5,998,463; Protein Science, 8 (10), 1930-1945 (1999), which in particular discloses the compound 3-isopropyl-4-(2-chlorophenyl)-1,4-dihydro-1-ethyl-2-methylpyridine; International Application Publication Nos. WO95/24391; WO97/09040; WO98/40353; WO01/23347; WO98/50359; and WO97/31901; European Application Publication No. EP 0 884 050; and J. Med. Chem., 41, 2934-2938 (1998).

One class of generally preferred glycogen phosphorylase inhibitors useful in such combinations comprises, for example, the compounds disclosed in European Patent Application Publication Number EP 1088824, and in commonly assigned International Application Publication No. WO 96/39384 and U.S. Pat. Nos. 6,107,329 and 6,277,877.

The compounds of Formula I, the prodrugs thereof, and the pharmaceutically acceptable salts of the compounds and prodrugs, are administered to a patient in need of treatment therewith in therapeutically effective amounts. The compounds can be administered alone or, preferably, as part of a pharmaceutically acceptable composition. In addition, the compounds or compositions can be administered all at once, as for example, by a bolus injection, multiple times, such as by a series of tablets, or delivered substantially uniformly over a period of time, as for example, using transdermal delivery. It is also noted that the dose of the compound can be varied over time.

In addition, the compounds of Formula I, the prodrugs thereof, and the pharmaceutically acceptable salts of the compounds and prodrugs, can be administered alone, in combination with other compounds of the present invention, or with other pharmaceutically active compounds. The other pharmaceutically active compounds can be intended to treat the same disease or condition as the compounds of the present invention or a different disease or condition. If the patient is to receive or is receiving multiple pharmaceutically active compounds, the compounds can be administered simultaneously, or sequentially in any order. For example, in the case of tablets, the active compounds may be found in one tablet or in separate tablets, which can be administered at once or sequentially. In addition, it should be recognized that the compositions may comprise different forms. For example, one or more compounds may be delivered via a tablet, while another is administered via injection or orally as a syrup. All combinations, delivery methods and administration sequences are intended to be embraced within the scope of the invention.

The compounds of Formula I, the prodrugs thereof, and the pharmaceutically acceptable salts of the compounds and prodrugs, and other pharmaceutically active agents, if desired, can be administered to a patient either orally, rectally, parenterally (for example, intravenously, intramuscularly, or subcutaneously), intracisternally, intravaginally, intraperitoneally, intravesically, locally (for example, powders, ointments or drops), or as a buccal or nasal spray.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Prevention of microorganism contamination of the compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and gelatin.

Solid dosage forms for oral administration include capsules, tablets, powders, and granules. In such solid dosage forms, the active compound is admixed with at least one inert customary pharmaceutical excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings and shells, such as enteric coatings and others well known in the art. They may also contain opacifying agents, and can also be of such composition that they release the active compound or compounds of this invention in a delayed manner. Examples of embedding compositions that can be used are polymeric substances and waxes. The active compounds of this invention can also be in micro-encapsulated form, if appropriate, with one or more of the above-entioned excipients.

Liquid dosage forms for oral administration include pharmaceutically acceptable emulsions, solutions, suspensions, syrups, and elixirs. In addition to the active compounds of this invention, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the active compound of this invention, may further comprise suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by mixing a compound of the present invention with suitable non-irritating excipients or carriers such as cocoa butter, polyethylene glycol or a suppository wax, which are solid at ordinary room temperature, but liquid at body temperature, and therefore, melt in the rectum or vaginal cavity thereby releasing the active component.

Dosage forms for topical administration of the compounds of Formula I, the prodrugs thereof, and the pharmaceutically acceptable salts of the compounds and prodrugs, may comprise ointments, powders, sprays and inhalants. The agent or agents are admixed under sterile conditions with a physiologically acceptable carrier, and any preservatives, buffers, or propellants that may be required. Opthalmic formulations, eye ointments, powders, and solutions are also intended to be within the scope of the present invention.

When treating any of the conditions, disorders and/or diseases previously disclosed herein, generally satisfactory results are obtained when the compounds of the Formula I, prodrugs, or pharmaceutically acceptable salts thereof are administered to mammals, including man or poultry, via either the oral or the parenteral route. Administration by the oral route is preferred, being more convenient and avoiding the possible pain and irritation of injection. However, in circumstances where the patient cannot swallow the medication, or absorption following oral administration is impaired, as by disease or other abnormality, it is essential that the drug be administered parenterally. By either route, the dosage is in the range of about 0.01 to about 100 mg/kg body weight of the subject per day, preferably about 0.1 to about 10 mg/kg body weight per day, administered singly or as a divided dose. However, the optimum dosage for the individual subject being treated will be determined by the person responsible for the treatment, generally smaller doses being administered initially and thereafter increasing increments made to determine the most suitable dosage. This will vary according to the particular compound employed and with the subject being treated.

The effective dosage of the compound employed may vary depending on the particular compound employed, the mode of administration, the condition being treated and the severity of the condition being treated.

Since one aspect of the present invention comprises the treatment of the disease/conditions with a combination of pharmaceutically active agents that may be administered separately, the invention further relates to combining separate pharmaceutical compositions in kit form.

The kit, according to the invention, comprises two separate pharmaceutical compositions: a compound of Formula I, or a prodrug thereof, or a pharmaceutically acceptable salt of the compound or prodrug; and an additional pharmaceutically active compound. The kit further comprises a container for containing the separate compositions, such as a divided bottle or a divided foil packet. Additional examples of containers include syringes, boxes, bags, and the like. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

One well known example of such a kit is a so-called blister pack. Blister packs are well known in the packaging industry and are being widely employed for the packaging of pharmaceutical unit dosage forms (tablets, capsules, and the like).

Blister packs generally comprise a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of compounds of the present invention can consist of one tablet or capsule, while a daily dose of the second compound can consist of several tablets or capsules and vice versa. The memory aid should reflect this and aid in correct administration of the active agents.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of Formula I of the invention may be named according to the IUPAC (International Union for Pure and Applied Chemistry) or CAS (Chemical Abstracts Service) nomenclature systems. In this specification and appendant claims, the compounds are named using the IUPAC system.

Throughout the present description, the following abbreviations or acronyms are used with the indicated meanings:

| | |
|---|---|
| ¹H | proton |
| ° C. | degrees Celsius |
| API | atmospheric pressure chemical ionization, positive ion mode |
| CDCl₃ | deuteriochloroform |
| dmso-d₆ | hexadeuteriodimethyl sulfoxide |
| g | grams |
| L | liters |
| mmol | millimoles |
| mL | milliliters |
| MS | mass spectrometry |
| NMR | nuclear magnetic resonance |
| psig | gas pressure expressed in pounds per square inch above |
| | atmospheric pressure |

The present invention is illustrated by the following Examples. It is to be understood, however, that the instant Examples are offered by way of illustrations of the invention and are not to be construed in any manner as limitations thereof. In the following examples, all solutions that are referred to as having been dried were dried over anhydrous magnesium sulfate unless otherwise noted. All evaporations were carried out on a rotary evaporator at ca. 30 Torr. Commercial reagents were used as received without additional purification unless otherwise noted. Solvents were commercial anhydrous grades and were used without further purification.

### Example 1a

4-[3-(6,7-Diethoxy-guinazolin-4-ylamino)-phenyl]-thiazole-2-carboxyic acid amide hydrochloride. A solution of 0.157 g (0.62 mmol) of 4-chloro-6,7-diethoxyquinazoline in 2.5 mL of ethanol was heated at reflux and treated with 0.136 g (0.62 mmol) of 4-(3-aminophenyl)-thiazole-2-carboxylic acid amide dissolved in 4 mL of ethanol added in a single portion. Heating was continued for 30 minutes, after which the reaction mixture was allowed to cool. The precipitated product was filtered, washed with ethanol and dried to afford 0.152 g (56%) of 4-[3-(6,7-diethoxy-quinazolin-4-ylamino)-phenyl]-thiazole-2-carboxylic acid amide hydrochloride, mp 264-266° C. ¹H NMR (dmso-d₆): δ11.40 (s, 1 H); 8.79 (s, 1 H); 8.42 (d, 1 H); 8.28 (s, 1 H); 8.24 (s, 1 H); 7.99 (d, 1 H); 7.93 (s, 1 H); 7.62 (m, 1 H); 7.55 (m, 1 H); 7.32 (m, 1 H); 4.23 (overlapping q, 4 H); 1.41 (overlapping t, 6 H). MS (API): m/z 436 (M+H⁺). Analysis (C₂₂H₂₁N₅O₃S·HCl): Calculated: C 55.99, H 4.70, N 14.84; Found: C 56.11, H 4.70, N 14.72.

### Example 1b

(6,7-Diethoxy-guinazolin-4-yl)-(3-pyridin-3-yl-phenyl)-amine hydrochloride. A solution of 0.042 g (0.25 mmol) of 3-pyridin-3-yl-phenylamine in 0.80 mL of 2-propanol was heated at reflux and treated with 0.050 g (0.20 mmol) of 4-chloro-6,7-diethoxyquinazoline added in a single portion. The mixture was heated for 2 hours, then cooled and filtered. The precipitate was washed with ethyl acetate and dried to afford 0.068 g (88%) of (6,7-diethoxy-quinazolin-4-yl)-(3-pyridin-3-yl-phenyl)-amine hydrochloride as colorless crystals, mp 255-256° C. ¹ H NMR (dmso-d₆): δ8.94 (s, 1 H); 8.79 (s, 1 H); 8.61 (d, 1 H); 8.23 (s, 1 H); 8.15 (d, 1 H); 8.06 (s, 1 H); 7.74-7.53 (m, 4 H); 4.23 (overlapping q, 4 H); 1.42 (overlapping t, 6 H). MS (API): m/z 387 (M+H⁺). Analysis (C₂₃H₂₂N₄O₂·HCl): Calculated: C 65.32, H 5.48, N 13.25; Found: C 65.72, H 5.45, N 12.86.

### Example 1c

2-[3-(6,7-Diethoxy-guinazolin-4-ylamino)-phenyl]-thiazole-4-carboxylic acid amide hydrochloride. A solution of 0.055 g (0.25 mmol) of 2-(3-aminophenyl)-thiazole-4-carboxylic acid amide in 1 mL of ethanol was heated at reflux and treated with 0.050 g (0.2 mmol) of 4-chloro-6,7-diethoxyquinazoline. The mixture was heated at reflux for 1 hour, then the precipitate was filtered, washed with ethanol and dried to afford 0.077 g (88%) of 2-[3-(6,7-diethoxy-quinazolin-4-ylamino)-phenyl]-thiazole-4-carboxylic acid amide hydrochloride as white crystals, mp 284-286° C. ¹H NMR (dmso-d₆): δ8.86 (s, 1 H); 8.34 (m, 2 H); 8.29 (m, 1 H); 7.97 (d, 1 H); 7.90 (m, 2 H); 7.70 (br, 1 H); 7.64 (t, 1 H); 7.35 (s, 1 H); 4.27 (overlapping q, 4 H); 1.46 (overlapping t, 6 H). MS (API): m/z 436 (M+H⁺). Analysis (C₂₂H₂₁N₅O₃S·HCl): Calculated: C 55.99, H 4.70, N 14.84; Found: C 55.69, H 4.64, N 14.62.

### Example 1d

(6,7-Diethoxy-quinazolin-4-yl)-[4-fluoro-3-(2-methyl-thiazol-4-yl)-phenyl]-amine hydrochloride. A solution of 0.052 g (0.25 mmol) of 4-fluoro-3-(2-methyl-thiazol-4-yl)-phenylamine in 0.8 mL of 2-propanol was heated at reflux and treated with 0.050 g (0.2 mmol) of 4-chloro-6,7-diethoxyquinazoline. The mixture was heated at reflux for 1 hour, then the precipitate was filtered, washed with 2-propanol and dried to afford 0.069 g (82%) of (6,7-diethoxy-quinazolin-4-yl)-[4-fluoro-3-(2-methyl-thiazol-4-yl)-phenyl]-amine hydrochloride as white crystals, mp 280-281° C. ¹ H NMR (dmso-d₆): δ8.80 (s, 1 H); 8.30 (m, 1 H); 8.21 (s, 1 H); 7.88 (s, 1 H); 7.75 (m, 1 H); 7.43 (t, 1 H); 7.32 (s, 1 H); 4.25 (overlapping q, 4 H); 2.69 (s, 3 H); 1.42 (overlapping t, 6 H). MS (API): m/z 436 (M+H⁺). Analysis (C₂₂H₂,FN₄O₂S·HCl): Calculated: C 57.32, H 4.81, N 12.15; Found: C 57.62, H 4.79, N 12.14.

### Example 1e

(2-Chloromethyl-6,7-diethoxy-quinazolin-4-yl)-[3-(2-methyl-thiazol-4-yl)-phenyl]-amine hydrochloride. A solution of 2.56 g of 4-chloro-2-chloromethyl-6,7-diethoxy-quinazoline and 1.85 g (9.7 mmol) of 3-(2-methyl-thiazol-4-yl)-phenylamine in 20 mL of 2-propanol was heated under reflux for 2 hours and then cooled. The precipitate was filtered, washed with 2-propanol and dried to afford 3.31 g (83%) of (2-chloromethyl-6,7-diethoxy-quinazolin-4-yl)-[3-(2-methyl-thiazol-4-yl)-phenyl]-amine hydrochloride, mp 252-256° C. ¹H NMR (dmso-d₆): δ8.28 (s, 1 H); 8.21 (s, 1 H); 7.91 (s, 1 H); 7.83 (d, 1 H); 7.76 (d, 1 H); 7.47 (t 1 H); 7.33 (s, 1 H); 4.79 (s, 2 H); 4.24 (q 2 H); 4.19 (q , 2 H); 2.68 (s, 3 H); 1.39 (overlapping t, 6 H). MS (API): m/z455, 457 (M+H⁺, Cl isotope pattern). Analysis (C₂₃H₂₃CIN₄O₂S·HCl): Calculated: C 56.21, H 4.92, N 11.40; Found: C 56.40, H 5.01, N 11.49.

### Example 1f

(6-Ethoxy-7-isopropoxy-guinazolin-4-yl)-[3-(2-methyl-thiazol-4-yl)-phenyl]-amine hydrochloride. A solution of 0.067 g (0.25 mmol) of 4-chloro-6-ethoxy-7-isopropoxy-quinazoline in 1 mL of ethanol was heated under reflux and treated with 0.057 g (0.30 10 mmol) of 3-(2-methyl-thiazol-4-yl)-phenylamine. The mixture was heated for 1 hour, then cooled and allowed to stand at 20° C. for 16 hours. The precipitate was filtered, washed with ethanol and dried to afford 0.093 g (88%) of (6-ethoxy-7-isopropoxy-quinazolin-4-yl)-[3-(2-methyl-thiazol-4-yl)-phenyl]-amine hydrochloride, mp 257-259° C. ¹H NMR (dmso-d₆): δ8.79 (s, 1 H); 8.16 (m, 2 H); 7.97 (s, 1 H), 7.83 (d, 1 H); 7.68 (d, 1 H); 7.50 (t, 1 H); 7.36 (s, 1 H); 4.77 (m, 1 H); 4.25 (q, 2 H); 2.69 (s, 3 H); 1.42 (t, 3 H); 1.37 (d, 6 H). MS (API): m/z 421 (M+H⁺). Analysis (C₂₃H₂₄N₄O₂S·HCl): Calculated: C 60.45, H 5.51, N 12.26; Found: C 60.37, H 5.38, N 11.87.

Examples 1g -1cg were prepared from the appropriate starting materials in a manner analogous to the methods of Examples 1a through 1f.

| Ex. | | | mp |
|---|---|---|---|
| # | Compound | Mass* | (° C.) |
| 1g | (6,7-Diethoxy-quinazolin-4-yl)-(3-[1,2,4]oxa- | 378 | 270 |
| | diazol-3-yl-phenyl)-amine | | |
| 1h | (6,7-Diethoxy-quinazolin-4-yl)-(3-oxazol-4-yl- | 377 | 250- |
| | phenyl)-amine | | 251 |
| 1i | (6,7-Diethoxy-quinazolin-4-yl)-[3-(2-methyl- | 407 | 259- |
| | thiazol-4-yl)-phenyl]-amine | | 260 |
| 1j | [3-(2-Amino-thiazol-4-yl)-phenyl]-(6,7- | 408 | 256- |
| | diethoxy-quinazolin-4-yl)-amine | | 259 |
| 1k | (6,7-Diethoxy-quinazolin-4-yl)-(3-thiazol-4-yl- | 393 | 265- |
| | phenyl)-amine | | 266 |
| 1l | (6,7-Diethoxy-quinazolin-4-yl)-[3-(2-methyl- | 391 | 276- |
| | oxazol-5-yl)-phenyl]-amine | | 277 |
| 1m | (6,7-Diethoxy-quinazolin-4-yl)-(3-pyrimidin-5- | 388 | 276- |
| | yl-phenyl)-amine | | 277 |
| 1n | (6,7-Diethoxy-quinazolin-4-yl)-[3-(2H-pyrazol- | 376 | 248- |
| | 3-yl)-phenyl]-amine | | 249 |
| 1o | (6,7-Diethoxy-quinazolin-4-yl)-(3-thiazol-5-yl- | 393 | 264- |
| | phenyl)-amine | | 265 |
| 1p | (6,7-Diethoxy-quinazolin-4-yl)-(3-thiazol-2-yl- | 393 | 251- |
| | phenyl)-amine | | 252 |
| 1q | (6,7-Dimethoxy-quinazolin-4-yl)-(3-thiazol-2- | 365 | 235- |
| | yl-phenyl)-amine | | 236 |
| 1r | (6,7-Diethoxy-quinazolin-4-yl)-(3-furan-2-yl- | 376 | 240- |
| | phenyl)-amine | | 244 |
| 1s | (6,7-Dimethoxy-quinazolin-4-yl)-(3-furan-2-yl- | 348 | 220- |
| | phenyl)-amine | | 224 |
| 1t | (6,7-Diethoxy-quinazolin-4-yl)-(3-oxazol-2-yl- | 377 | 256- |
| | phenyl)-amine | | 258 |
| 1u | (6,7-Diethoxy-quinazolin-4-yl)-[3-(2H- | 377 | 270- |
| | [1,2,4]triazol-3-yl)-phenyl]-amine | | 273 |
| 1v | (6,7-Diethoxy-quinazolin-4-yl)-[3-(2-methyl- | 391 | |
| | 2H-[1,2,4]triazol-3-yl)-phenyl]-amine | | |
| 1w | (6,7-Dimethoxy-quinazolin-4-yl)-[3-(2-methyl- | 379 | 257- |
| | thiazol-4-yl)-phenyl]-amine | | 258 |
| 1x | (6,7-Diethoxy-quinazolin-4-yl)-[3-(2-ethoxy- | 437 | 243- |
| | thiazol-4-yl)-phenyl]-amine | | 247 |
| 1y | (6,7-Diethoxy-quinazolin-4-yl)-[3-(2-methoxy- | 423 | 228- |
| | thiazol-4-yl)-phenyl]-amine | | 231 |
| 1z | (6,7-Diethoxy-quinazolin-4-yl)-[3-(2-methoxy- | 437 | 232- |
| | methyl-thiazol-4-yl)-phenyl]-amine | | 233 |
| 1aa | (6,7-Diethoxy-2-ethyl-quinazolin-4-yl)-[3-(2- | 435 | 243- |
| | methyl-thiazol-4-yl)-phenyl]-amine | | 244 |
| 1ab | (6,7-Diethoxy-2-methyl-quinazolin-4-yl)-[3-(2- | 421 | 249- |
| | methyl-thiazol-4-yl)-phenyl]-amine | | 250 |
| 1ac | (6,7-Diethoxy-2-isopropyl-quinazolin-4-yl)-[3- | 449 | 190- |
| | (2-methyl-thiazol-4-yl)-phenyl]-amine | | 191 |
| 1ad | (6,7-Diethoxy-quinazolin-4-yl)-[3-(2-methyl- | 422 | 252- |
| | amino-thiazol-4-yl)-phenyl]-amine | | 253 |
| 1ae | (6,7-Diethoxy-2-propyl-quinazolin-4-yl)-[3-(2- | 449 | 239- |
| | methyl-thiazol-4-yl)-phenyl]-amine | | 240 |
| 1af | (6,7-Diethoxy-2-methoxymethyl-quinazolin-4- | 451 | 141- |
| | yl)-[3-(2-methyl-thiazol-4-yl)-phenyl]-amine | | 143 |
| 1ag | (2-Cyclopropyl-6,7-diethoxy-quinazolin-4-yl)- | 447 | 194 |
| | [3-(2-methyl-thiazol-4-yl)-phenyl]-amine | | |
| 1ah | (6,7-Diethoxy-2-methylsulfanylmethyl- | 467 | |
| | quinazolin-4-yl)-[3-(2-methyl-thiazol-4-yl)- | | |
| | phenyl]-amine | | |
| 1ai | (6,7-Diethoxy-2-methanesulfonylmethyl- | 499 | |
| | quinazolin-4-yl)-[3-(2-methyl-thiazol-4-yl)- | | |
| | phenyl]-amine | | |
| 1aj | (6,7-Diethoxy-2-isobutyl-quinazolin-4-yl)-[3- | 463 | |
| | (2-methyl-thiazol-4-yl)-phenyl]-amine | | |
| 1ak | (6-Ethoxy-7-methoxy-quinazolin-4-yl)-[3-(2- | 393 | 254- |
| | methyl-thiazol-4-yl)-phenyl]-amine | | 257 |
| 1al | (7-Ethoxy-6-methoxy-quinazolin-4-yl)-[3-(2- | 393 | 259- |
| | methyl-thiazol-4-yl)-phenyl]-amine | | 261 |
| 1am | [2-(2-Chloro-ethyl)-6,7-diethoxy-quinazolin- | 469 | 229- |
| | 4-yl]-[3-(2-methyl-thiazol-4-yl)-phenyl]-amine | | 230 |
| 1an | {6,7-Diethoxy-4-[3-(2-methyl-thiazol-4-yl)- | 465 | |
| | phenylamino]-quinazolin-2-yl}-acetic acid | | |
| 1ao | {6,7-Diethoxy-4-[3-(2-methyl-thiazol-4-yl)- | 493 | |
| | phenylamino]-quinazolin-2-yl}-acetic acid | | |
| | ethyl ester | | |
| 1ap | (2-Butyl-6,7-diethoxy-quinazolin-4-yl)-[3-(2- | 463 | |
| | methyl-thiazol-4-yl)-phenyl]-amine | | |
| 1ag | {6,7-Diethoxy-4-[3-(2-methyl-thiazol-4-yl)- | 479 | |
| | phenylamino]-quinazolin-2-yl}-acetic acid | | |
| | methyl ester | | |
| 1ar | {6,7-Diethoxy-4-[3-(2-methyl-thiazol-4-yl)- | 465 | |
| | phenylamino]-quinazolin-2-yl}-acetic acid | | |
| 1as | [4-Chloro-3-(2-methyl-thiazol-4-yl)-phenyl]- | 441 | 275- |
| | (6,7-diethoxy-quinazolin-4-yl)-amine | | 276 |
| 1at | (6,7-Diethoxy-quinazolin-4-yl)-[2-fluoro-5-(2- | 425 | 267- |
| | methyl-thiazol-4-yl)-phenyl]-amine | | 268 |
| 1au | [2-Chloro-5-(2-methyl-thiazol-4-yl)-phenyl]- | 441 | 261 |
| | (6,7-diethoxy-quinazolin-4-yl)-amine | | |
| 1av | (6,7-Diethoxy-quinazolin-4-yl)-[2-methyl-5- | 421 | 231- |
| | (2-methyl-thiazol-4-yl)-phenyl]-amine | | 232 |
| 1aw | (6,7-Diethoxy-quinazolin-4-yl)-[2-methoxy-5- | 437 | 194- |
| | (2-methyl-thiazol-4-yl)-phenyl]-amine | | 197 |
| 1ax | (6,7-Diethoxy-quinazolin-4-yl)-[3-(2,5- | 421 | |
| | dimethyl-thiazol-4-yl)-phenyl]-amine | | |
| 1ay | [2-(3-Chloro-propyl)-6,7-diethoxy-quinazolin- | 483 | 185- |
| | 4-yl]-[3-(2-methyl-thiazol-4-yl)-phenyl]-amine | | 188 |
| 1az | (7-Isopropoxy-6-methoxy-quinazolin-4-yl)-[3- | 407 | 259- |
| | (2-methyl-thiazol-4-yl)-phenyl]-amine | | 260 |
| 1ba | (6,7-Diethoxy-quinazolin-4-yl)-[3-(4-methyl- | 407 | 270- |
| | thiazol-2-yl)-phenyl]-amine | | 271 |
| 1bb | (7-sec-Butoxy-6-methoxy-quinazolin-4-yl)-[3- | 421 | 222- |
| | (2-methyl-thiazol-4-yl)-phenyl]-amine | | 224 |
| 1bc | (6-Methoxy-7-propoxy-quinazolin-4-yl)-[3-(2- | 407 | 252- |
| | methyl-thiazol-4-yl)-phenyl]-amine | | 253 |
| 1bd | {4-[3-(6,7-Diethoxy-quinazolin-4-ylamino)- | 423 | 227- |
| | phenyl]-thiazol-2-yl}-methanol | | 229 |
| 1be | (6-Ethoxy-7-propoxy-quinazolin-4-yl)-[3-(2- | 421 | 238- |
| | methyl-thiazol-4-yl)-phenyl]-amine | | 241 |
| 1bf | (6,7-Diethoxy-quinazolin-4-yl)-[2-ethoxy-5- | 451 | 185 |
| | (2-methyl-thiazol-4-yl)-phenyl]-amine | | (dec) |
| 1bg | (6,7-Diethoxy-quinazolin-4-yl)-[2-ethyl-5-(2- | 435 | 179- |
| | methyl-thiazol-4-yl)-phenyl]-amine | | 183 |
| 1bh | (6,7-Diethoxy-quinazolin-4-yl)-(3-pyridin-2-yl- | 387 | 262 |
| | phenyl)-amine | | |
| 1bi | 4-[3-(6-Ethoxy-7-propoxy-quinazolin-4- | 450 | |
| | ylamino)-phenyl]-thiazole-2-carboxylic acid | | |
| | amide | | |
| 1bj | 4-[3-(6-Ethoxy-7-isopropoxy-quinazolin- | 450 | |
| | 4-ylamino)-phenyl]-thiazole-2-carboxylic | | |
| | acid amide | | |
| 1bk | 4-[3-(7-sec-Butoxy-6-methoxy-quinazolin- | 450 | |
| | 4-ylamino)-phenyl]-thiazole-2-carboxylic | | |
| | acid amide | | |
| 1bl | (6-Ethoxy-7-propoxy-quinazolin-4-yl)-[4- | 439 | |
| | fluoro-3-(2-methyl-thiazol-4-yl)-phenyl]-amine | | |
| 1bm | (6-Ethoxy-7-isopropoxy-quinazolin-4-yl)-[4- | 439 | |
| | fluoro-3-(2-methyl-thiazol-4-yl)-phenyl]-amine | | |
| 1bn | 4-[5-(7-sec-Butoxy-6-methoxy-quinazolin-4- | 468 | |
| | ylamino)-2-fluoro-phenyl]-thiazole-2- | | |
| | carboxylic acid amide | | |
| 1bo | 4-[5-(6-Ethoxy-7-isopropoxy-quinazolin-4- | 468 | |
| | ylamino)-2-fluoro-phenyl]-thiazole-2- | | |
| | carboxylic acid amide | | |
| 1bp | {4-[3-(6-Ethoxy-7-isopropoxy-quinazolin- | 437 | |
| | 4-ylamino)-phenyl]-thiazol-2-yl}-methanol | | |
| 1bq | 2-[3-(6-Ethoxy-7-isopropoxy-quinazolin-4- | 450 | |
| | ylamino)-phenyl]-thiazole-4-carboxylic | | |
| | acid amide | | |
| 1br | 4-[3-(7-sec-Butoxy-6-ethoxy-quinazolin-4- | 464 | |
| | ylamino)-phenyl]-thiazole-2-carboxylic | | |
| | acid amide | | |
| 1bs | {4-[3-(7-sec-Butoxy-6-ethoxy-quinazolin- | 451 | |
| | 4-ylamino)-phenyl]-thiazol-2-yl}-methanol | | |
| 1bt | 2-[3-(7-sec-Butoxy-6-ethoxy-quinazolin-4- | 464 | |
| | ylamino)-phenyl]-thiazole-4-carboxylic | | |
| | acid amide | | |
| 1bu | 4-[3-(6,7-Diethoxy-2-methoxymethyl- | 480 | |
| | quinazolin-4-ylamino)-phenyl]-thiazole-2- | | |
| | carboxylic acid amide | | |
| 1bv | {4-[3-(6,7-Diethoxy-2-methoxymethyl- | 467 | |
| | quinazolin-4-ylamino)-phenyl]-thiazol-2-yl}- | | |
| | methanol | | |
| 1bw | (6,7-Diethoxy-2-methoxymethyl-quinazolin-4-yl)- | 431 | |
| | (3-pyridin-2-yl-phenyl)-amine | | |
| 1bx | (6,7-Diethoxy-quinazolin-4-yl)-(3-quinolin-3- | 437 | |
| | yl-phenyl)-amine | | |
| 1by | (6,7-Diethoxy-quinazolin-4-yl)-[3-(2-methyl- | 390 | 272- |
| | oxazol-4-yl)-phenyl]-amine | | 274 |
| 1bz | (6,7-Diethoxy-quinazolin-4-yl)-(3-pyrimidin-4- | 388 | |
| | yl-phenyl)-amine | | |
| 1ca | 4-[3-(6-Ethoxy-7-isopropoxy-2-methoxy- | 494 | |
| | methyl-quinazolin-4-ylamino)-phenyl]-thiazole- | | |
| | 2-carboxylic acid amide | | |
| 1cb | {4-[3-(6-Ethoxy-7-isopropoxy-2-methoxy- | 481 | |
| | methyl-quinazolin-4-ylamino)-phenyl]-thiazol-2- | | |
| | yl}-methanol | | |
| 1cc | (2-Chloromethyl-6-ethoxy-7-isopropoxy- | 469 | |
| | quinazolin-4-yl)-[3-(2-methyl-thiazol-4-yl)- | | |
| | phenyl]-amine | | |
| 1cd | 2-[3-(6,7-Diethoxy-quinazolin-4-ylamino)- | 418 | |
| | phenyl]-thiazole-4-carbonitrile | | |
| 1ce | (6-Ethoxy-7-isopropoxy-quinazolin-4-yl)- | 407 | |
| | (3-thiazol-5-yl-phenyl)-amine | | |
| 1cf | 2-[3-(6-Ethoxy-7-isopropoxy-quinazolin-4- | 432 | |
| | ylamino)-phenyl]-thiazole-4-carbonitrile | | |
| 1cg | {4-[3-(2-Chloromethyl-6-ethoxy-7-iso- | 485 | |
| | propoxy-quinazolin-4-ylamino)-phenyl]-thiazol- | | |
| | 2-yl}-methanol | | |

*The mass given is that of the most abundant parent ion, MH⁺.

### Example 2a

(6,7-Diethoxy-guinazolin-4-yl)-[3-(2-methylamino-thiazol-4-yl)-phenyl]-amine hydrobromide.

Step A: 1-[3-(6,7-Diethoxy-cluinazolin-4-ylamino)-phenyl]-ethanone hydrochloride. A solution of 4.30 g (16.9 mmol) of 4-chloro-6,7-diethoxyquinazoline in 80 mL of 2-propanol was heated at reflux and treated with 2.51 g (18.6 mmol) of 3-aminoacetophenone added in a single portion. Heating was continued for 1 hour, after which the reaction mixture was allowed to cool. The precipitated product was filtered, washed with 2-propanol and dried to afford 5.19 g (87%) of 1-[3-(6,7-diethoxy-quinazolin-4-ylamino)-phenyl]-ethanone hydrochloride, mp 258-260° C. ¹H NMR (dmso-d₆): δ8.81 (s, 1 H); 8.18 (m, 2 H); 7.97 (d, 1 H); 7.88 (d, 1 H); 7.62 (t, 1 H); 7.27 (s, 1 H); 4.24 (overlapping q, 4 H); 2.59 (s, 3 H); 1.43 (overlapping t, 6 H). MS (API): m/z 352 (M+H⁺). Analysis (C₂₀H₂₁N₃O₃·HCl): Calculated: C 61.93, H 5.72, N 10.83; Found: C 62.07, H 5.78, N 10.86.

Step 2: 2-Bromo-1-[3-(6,7-diethoxy-guinazolin-4-ylamino)-phenyl]-ethanone hydrobromide. A solution of 3.53 g (10 mmol) of 1-[3-(6,7-diethoxy-quinazolin-4-ylamino)-phenyl]-ethanone hydrochloride in 45 mL of acetic acid and 15 mL of 48% aqueous hydrobromic acid was heated to reflux and treated with 10 mL of a 1 molar solution of bromine in acetic acid. After 20 minutes the color had been discharged and the reaction was cooled and filtered to afford 2.79 g (65%) of 2-bromo-1-[3-(6,7-diethoxy-quinazolin-4-ylamino)-phenyl]-ethanone hydrobromide, mp>315° C. ¹H NMR (dmso-d₆): δ8.84 (s, 1 H); 8.20 (s, 1 H); 8.06 (s, 1 H); 7.97 (m, 2 H); 7.66 (t, 1 H); 7.22 (s, 1 H); 4.95 (s, 2 H); 4.25 (overlapping q, 4 H); 1.43 (overlapping q, 6 H). MS (API): m/z 430, 432 (M+H⁺). Analysis (C₂₀H₂₀BrN₃O₃·HBr): Calculated: C 46.99, H 4.14, N 8.22; Found: C 46.87, H 4.01, N 8.29.

Step 3: A mixture of 0.432 g (1.0 mmol) of 2-bromo-1-[3-(6,7-diethoxy-quinazolin-4-ylamino)-phenyl]-ethanone hydrobromide and 0.022 g (0.25 mmol) of N-methylthiourea in 2 mL of ethanol was heated at reflux for 2 hours. The reaction mixture was filtered while still hot. The precipitate was washed with ethanol and dried to afford 0.075 g (74%) of (6,7-diethoxy-quinazolin-4-yl)-[3-(2-methylamino-thiazol-4-yl)-phenyl]-amine dihydrobromide, mp 252-253° C. ¹H NMR (dmso-d₆): δ8.83 (s, 1 H); 8.12 (s, 1 H); 7.99 (s, 1 H); 7.75 (d, 1 H); 7.60 (d, 1 H); 7.50 (t, 1 H); 7.23 (s, 1 H); 7.16 (s, 1 H); 4.24 (overlapping q, 4 H); 2.90 (s, 3 H); 1.43 (overlapping q, 6 H). MS (API): m/z 422 (M+H⁺). Analysis (C₂₂H₂₃N₅O₂S·2HBr): Calculated: C 45.30; H 4.32; N 12.01; Found: C 45.40; H 4.70; N 11.81.

Examples 2b - 2d were prepared from the appropriate starting materials in a manner analogous to the method of Example 2a.

| Ex. | | | mp |
|---|---|---|---|
| # | Compound | Mass* | (° C.) |
| 2b | (6,7-Diethoxy-quinazolin-4-yl)-[3-(2-dimethyl- | 436 | 195- |
| | amino-thiazol-4-yl)-phenyl]-amine | | 198 |
| 2c | (6,7-Diethoxy-quinazolin-4-yl)-[3-(2-hydrazino- | 423 | 239- |
| | thiazol-4-yl)-phenyl]-amine | | 242 |
| 2d | [3-(2-Cyclopropyl-thiazol-4-yl)-phenyl]-(6,7- | 433 | 227- |
| | diethoxy-quinazolin-4-yl)-amine | | 229 |

*The mass given is that of the most abundant parent ion, MH⁺.

### Example 3a

(2-Dimethylaminomethyl-6,7-diethoxy-guinazolin-4-yl)-[3-(2-methyl-thiazol-4-yl)-phenyl]-amine. A suspension of 0.113 g (0.25 mmol) of (2-chloromethyl-6,7-diethoxy-quinazolin-4-yl)-[3-(2-methyl-thiazol-4-yl)-phenyl]-amine hydrochloride in 5 mL of 1,4-dioxane was saturated with dimethylamine gas. The mixture was heated at 80° C. in a sealed tube for 6 hours. The mixture was cooled, filtered, and the precipitate was washed with ethanol and dried to afford 0.094 g (81%) of (2-dimethylaminomethyl-6,7-diethoxy-quinazolin-4-yl)-[3-(2-methyl-thiazol-4-yl)-phenyl]-amine, mp 170-172° C. ¹H NMR (dmso-d₆): δ9.48 (s, 1 H); 8.56 (s, 1 H); m7.94 (d, 1 H); 7.91 (s, 1 H); 7.86 (s, 1 H); 7.59 (d, 1 H); 7.38 (t, 1 H); 7.14 (s, 1 H); 4.19 (overlapping q, 4 H); 3.57 (s, 2 H); 3.30 (s, 3 H); 2.28 (s, 6 H); 1.41 (overlapping t, 6 H). MS (API): m/z 464 (M+H⁺). Analysis (C₂₅H₂₉N₅O₂S): Calculated: C 64.77, H 6.31, N 15.11; Found: C 64.44, H 6.28, N 15.32.

Examples 3b - 3bf were prepared from the appropriate starting materials in a manner analogous to the method of Example 3a.

| Ex. # | Compound | Mass* | mp (° C.) |
|---|---|---|---|
| 3b | (2-Diethylaminomethyl-6,7-diethoxy-quinazolin-4-yl)-[3-(2- | 492 | |
| | methyl-thiazol-4-yl)-phenyl]-amine | | |
| 3c | (6,7-Diethoxy-2-ethylaminomethyl-quinazolin-4-yl)-[3-(2- | 464 | 235-237 |
| | methyl-thiazol-4-yl)-phenyl]-amine | | |
| 3d | (6,7-Diethoxy-2-morpholin-4-ylmethyl-quinazolin-4-yl)-[3-(2- | 506 | |
| | methyl-thiazol-4-yl)-phenyl]-amine | | |
| 3e | (6,7-Diethoxy-2-piperidin-1-ylmethyl-quinazolin-4-yl)-[3-(2- | 504 | |
| | methyl-thiazol-4-yl)-phenyl]-amine | | |
| 3f | (6,7-Diethoxy-2-pyrrolidin-1-ylmethyl-quinazolin-4-yl)-[3-(2- | 490 | 232-235 |
| | methyl-thiazol-4-yl)-phenyl]-amine | | |
| 3g | [6,7-Diethoxy-2-(4-methyl-piperazin-1-ylmethyl)-quinazolin- | 519 | 93-96 |
| | 4-yl]-[3-(2-methyl-thiazol-4-yl)-phenyl]-amine | | |
| 3h | (6,7-Diethoxy-2-methylaminomethyl-quinazolin-4-yl)-[3-(2- | 450 | |
| | methyl-thiazol-4-yl)-phenyl]-amine | | |
| 3i | (2-Aminomethyl-6,7-diethoxy-quinazolin-4-yl)-[3-(2-methyl- | 436 | 115-118 |
| | thiazol-4-yl)-phenyl]-amine | | |
| 3j | [2-(2-Amino-ethyl)-6,7-diethoxy-quinazolin-4-yl]-[3-(2- | 450 | |
| | methyl-thiazol-4-yl)-phenyl]-amine | | |
| 3k | (6,7-Diethoxy-2-thiazolidin-3-ylmethyl-quinazolin-4-yl)-[3-(2- | 508 | |
| | methyl-thiazol-4-yl)-phenyl]-amine | | |
| 3l | 4-[3-(6,7-Diethoxy-2-thiazolidin-3-ylmethyl-quinazolin-4- | 537 | |
| | ylamino)-phenyl]-thiazole-2-Carboxylic acid amide | | |
| 3m | 4-[5-(6,7-Diethoxy-2-thiazolidin-3-ylmethyl-quinazolin-4- | 555 | |
| | ylamino)-2-fluoro-phenyl]-thiazole-2-carboxylic acid amide | | |
| 3n | (6,7-Diethoxy-2-piperazin-1-ylmethyl-quinazolin-4-yl)-[3-(2- | 505 | |
| | methyl-thiazol-4-yl)-phenyl]-amine | | |
| 3o | 4-[5-(2-Dimethylaminomethyl-6,7-diethoxy-quinazolin-4- | 511 | |
| | ylamino)-2-fluoro-phenyl]-thiazole-2-carboxylic acid amide | | |
| 3p | 4-[3-(2-Dimethylaminomethyl-6,7-diethoxy-quinazolin-4- | 493 | |
| | ylamino)-phenyl]-thiazole-2-carboxylic acid amide | | |
| 3q | (2-Dimethylaminomethyl-6,7-diethoxy-quinazolin-4-yl)-[4- | 482 | |
| | fluoro-3-(2-methyl-thiazol-4-yl)-phenyl]-amine | | |
| 3r | 4-[3-(6,7-Diethoxy-2-morpholin-4-ylmethyl-quinazolin-4- | 535 | |
| | ylamino)-phenyl]-thiazole-2-carboxylic acid amide | | |
| 3s | 4-[5-(6,7-Diethoxy-2-morpholin-4-ylmethyl-quinazolin-4- | 553 | |
| | ylamino)-2-fluoro-phenyl]-thiazole-2-carboxylic acid amide | | |
| 3t | (6,7-Diethoxy-2-morpholin-4-ylmethyl-quinazolin-4-yl)-[4- | 524 | |
| | fluoro-3-(2-methyl-thiazol-4-yl)-phenyl]-amine | | |
| 3u | 2-[3-(6,7-Diethoxy-2-thiazolidin-3-ylmethyl-quinazolin-4- | 537 | |
| | ylamino)-phenyl]-thiazole-4-carboxylic acid amide | | |
| 3v | (6,7-Diethoxy-2-thiazolidin-3-ylmethyl-quinazolin-4-yl)-[3-(4- | 508 | |
| | methyl-thiazol-2-yl)-phenyl]-amine | | |
| 3w | (6,7-Diethoxy-2-thiazolidin-3-ylmethyl-quinazolin-4-yl)-[4- | 526 | |
| | fluoro-3-(2-methyl-thiazol-4-yl)-phenyl]-amine | | |
| 3x | (6,7-Diethoxy-2-thiomorpholin-4-ylmethyl-quinazolin-4-yl)- | 540 | |
| | [4-fluoro-3-(2-methyl-thiazol-4-yl)-phenyl]-amine | | |
| 3y | 4-[5-(6,7-Diethoxy-2-thiomorpholin-4-ylmethyl-quinazolin-4- | 569 | |
| | ylamino)-2-fluoro-phenyl]-thiazole-2-carboxylic acid amide | | |
| | ylamino)-2-fluoro-phenyl]-thiazole-2-carboxylic acid amide | | |
| 3z | 4-[3-(6,7-Diethoxy-2-thiomorpholin-4-ylmethyl-quinazolin-4- | 551 | |
| | ylamino)-phenyl]-thiazole-2-carboxylic acid amide | | |
| 3aa | 4-[3-(6,7-Diethoxy-2-pyrazol-1-ylmethyl-quinazolin-4- | 516 | |
| | ylamino)-phenyl]-thiazole-2-carboxylic acid amide | | |
| 3ab | (6,7-Diethoxy-2-pyrazol-1-ylmethyl-quinazolin-4-yl)-[3-(2- | 487 | |
| | methyl-thiazol-4-yl)-phenyl]-amine | | |
| 3ac | (6,7-Diethoxy-2-imidazol-1-ylmethyl-quinazolin-4-yl)-[3-(2- | 487 | 207-210 |
| | methyl-thiazol-4-yl)-phenyl]-amine | | |
| 3ad | (6,7-Diethoxy-2-thiomorpholin-4-ylmethyl-quinazolin-4-yl)- | 522 | |
| | [3-(2-methyl-thiazol-4-yl)-phenyl]-amine | | |
| 3ae | 4-[3-(6,7-Diethoxy-2-piperidin-1-ylmethyl-quinazolin-4- | 533 | |
| | ylamino)-phenyl]-thiazole-2-carboxylic acid amide | | |
| 3af | 4-[5-(6,7-Diethoxy-2-piperidin-1-ylmethyl-quinazolin-4- | 551 | |
| | ylamino)-2-fluoro-phenyl]-thiazole-2-carboxylic acid amide | | |
| 3ag | (6,7-Diethoxy-2-piperidin-1-ylmethyl-quinazolin-4-yl)-[4- | 522 | |
| | fluoro-3-(2-methyl-thiazol-4-yl)-phenyl]-amine | | |
| 3ah | 4-[3-(6,7-Diethoxy-2-pyrrolidin-1-ylmethyl-quinazolin-4- | 519 | |
| | ylamino)-phenyl]-thiazole-2-carboxylic acid amide | | |
| 3ai | 4-[5-(6,7-Diethoxy-2-imidazol-1-ylmethyl-quinazolin-4- | 534 | |
| | ylamino)-2-fluoro-phenyl]-thiazole-2-carboxylic acid amide | | |
| 3aj | 4-{5-[6,7-Diethoxy-2-(4-methyl-piperazin-1-ylmethyl)- | 566 | |
| | quinazolin-4-ylamino]-2-fluoro-phenyl}-thiazole-2-carboxylic | | |
| | acid amide | | |
| 3ak | (6,7-Diethoxy-2-pyrrolidin-1-ylmethyl-quinazolin-4-yl)-[4- | 508 | |
| | fluoro-3-(2-methyl-thiazol-4-yl)-phenyl]-amine | | |
| 3al | 4-[5-(6,7-Diethoxy-2-pyrrolidin-1-ylmethyl-quinazolin-4- | 537 | |
| | ylamino)-2-fluoro-phenyl]-thiazole-2-carboxylic acid amide | | |
| 3am | {4-[3-(2-Dimethylaminomethyl-6,7-diethoxy-quinazolin-4- | 480 | |
| | ylamino)-phenyl]-thiazol-2-yl}-methanol | | |
| 3an | [2-(2-Dimethylamino-ethyl)-6,7-diethoxy-quinazolin-4-yl]-[3- | 478 | 162-166 |
| | (2-methyl-thiazol-4-yl)-phenyl]-amine | | |
| 3ao | {4-[3-(6,7-Diethoxy-2-morpholin-4-ylmethyl-quinazolin-4- | 522 | |
| | ylamino)-phenyl]-thiazol-2-yl}-methanol | | |
| 3ap | 2-[3-(6,7-Diethoxy-2-morpholin-4-ylmethyl-quinazolin-4- | 535 | |
| | ylamino)-phenyl]-thiazole-5-carboxylic acid amide | | |
| 3aq | (4-{3-[6,7-Diethoxy-2-(4-methyl-Piperazin-1-ylmethyl)- | 535 | |
| | quinazolin-4-ylamino]-phenyl}-thiazol-2-yl)-methanol | | |
| 3ar | 2-{3-[6,7-Diethoxy-2-(4-methyl-piperazin-1-ylmethyl)- | 548 | |
| | quinazolin-4-ylamino]-phenyl}-thiazole-5-carboxylic acid | | |
| | amide | | |
| 3as | 2-[3-(6,7-Diethoxy-2-imidazol-1-ylmethyl-quinazolin-4- | 516 | |
| | ylamino)-phenyl]-thiazole-4-carboxylic acid amide | | |
| 3at | {4-[3-(6,7-Diethoxy-2-imidazol-1-ylmethyl-quinazolin-4- | 503 | |
| | ylamino)-phenyl]-thiazol-2-yl}-methanol | | |
| 3au | [6,7-Diethoxy-2-(2-morpholin-4-yl-ethyl)-quinazolin-4-yl]-[3- | 520 | |
| | (2-methyl-thiazol-4-yl)-phenyl]-amine | | |
| 3av | 4-{3-[6,7-Diethoxy-2-(2-morpholin-4-yl-ethyl)-quinazolin-4- | 549 | |
| | ylamino]-phenyl]-thiazole-2-carboxylic acid amide | | |
| 3aw | 4-[3-(6,7-Diethoxy-2-imidazol-1-ylmethyl-quinazolin-4- | 516 | |
| | ylamino)-phenyl]-thiazole-2-carboxylic acid amide | | |
| 3ax | (6-Ethoxy-7-isopropoxy-2-morpholin-4-ylmethyl-quinazolin- | 520 | |
| | 4-yl)-[3-(2-methyl-thiazol-4-yl)-phenyl]-amine | | |
| 3ay | {4-[3-(6-Ethoxy-7-isopropoxy-2-morphlin-4-ylmethyl- | 536 | |
| | quinazolin-4-ylamino)-phenyl]-thiazol-2-yl}-methanol | | |
| 3az | 2-[3-(6-Ethoxy-7-isopropoxy-2-morpholin-4-ylmethyl- | 549 | |
| | quinazolin-4-ylamino)-phenyl]-thiazole-4-carboxylic acid | | |
| | amide | | |
| 3ba | (6-Ethoxy-2-imidazol-1-ylmethyl-7-isopropoxy-quinazolin-4- | 501 | |
| | yl)-[3-(2-methyl-thiazol-4-yl)-phenyl]-amine | | |
| 3bb | 4-[3-(6-Ethoxy-2-imidazol-1-ylmethyl-7-isopropoxy- | 530 | |
| | quinazolin-4-ylamino)-phenyl]-thiazole-2-carboxylic acid | | |
| | amide | | |
| 3bc | {4-[3-(6-Ethoxy-2-imidazol-1-ylmethyl-7-isopropoxy- | 517 | |
| | quinazolin-4-ylamino)-phenyl]-thiazol-2-yl}-methanol | | |
| 3bd | 2-[3-(6-Ethoxy-2-imidazol-1-ylmethyl-7-isopropoxy- | 530 | |
| | quinazolin-4-ylamino)-phenyl]-thiazole-4-carboxylic acid | | |
| | amide | | |
| 3be | 4-[3-(2-Dimethylaminomethyl-6-ethoxy-7-isopropoxy- | 507 | |
| | quinazolin-4-ylamino)-phenyl]-thiazole-2-carboxylic acid | | |
| | amide | | |
| 3bf | {4-[3-(2-Dimethylaminomethyl-6-ethoxy-7-isopropoxy- | 494 | |
| | quinazolin-4-ylamino)-phenyl]-thiazol-2-yl}-methanol | | |

*The mass given is that of the most abundant parent ion, MH⁺.

### Preparation 1a

4-Chloro-6,7-diethoxyguinazoline. A solution of 8.45 g of 6,7-diethoxy-3H-quinazolin-4-one in 50 mL of phosphorus oxychloride was heated at reflux for 2 hours. The mixture was then cooled and concentrated. The residue was added to a solution of 120 g of sodium carbonate in 300 mL of water and 300 mL of ethyl acetate. The mixture was stirred for 15 minutes, then the ethyl acetate was washed with brine, dried, and concentrated. The residue was crystallized from 1-chlorobutane to give 8.02 g (88%) of 4-chloro-6,7-diethoxyquinazoline, mp 141-143° C. ¹H NMR (CDCl₃): δ8.83 (s, 1 H); 7.36 (s, 1 H); 7.30 (s, 1 H); 4.27 (overlapping q, 4 H); 1.56 (overlapping t, 6 H). MS (API): m/z 253, 255 (M+H⁺, chlorine isotope pattern).

### Preparation 1b

4-Chloro-2-chloromethyl-6,7-diethoxy-quinazoline. A mixture of 2.50 g (8.8 mmol) of 2-chloromethyl-6,7-diethoxy-3H-quinazolin-4-one in 30 mL of phosphorus oxychloride was heated at reflux for 2 hours. The mixture was then cooled and added dropwise to a mixture of 2 g of tribasic sodium phosphate and 200 mL of water with stirring. The mixture was maintained at pH 5 by addition of 6 molar aqueous sodium hydroxide as needed, while the temperature was maintained below 25° C. by addition of ice. When the addition was complete the mixture was extracted with chloroform. The combined chloroform extracts were washed three times with water, brine, dried, and concentrated to afford 2.56 g (99%) of 4-chloro-2-chloromethyl-6,7-diethoxy-quinazoline, mp 109-112° C. ¹H NMR (CDCl₃): δ7.36 (s, 1 H); 7.33 (s, 1 H); 4.78 (s, 2 H); 4.26 (overlapping q, 4 H); 1.56 (overlapping t, 6 H). MS (API): m/z 301, 303, 305 (M+H⁺, 2 chlorine isotope pattern).

### Preparation 1c

4-Chloro-6-ethoxy-7-isopropoxy-quinazoline. A solution of 0.744 g (3 mmol) of 6-ethoxy-7-isopropoxy-3H-quinazolin-4-one and 0.150 g (1.1 mmol) of triethylamine hydrochloride in 3 mL of phosphorus oxychloride was heated at 120° C. for 3 hours. The mixture was concentrated and the residue was treated with ethyl acetate and 1 molar aqueous tribasic sodium phosphate and stirred for 30 minutes. The ethyl acetate was separated and the washing was repeated. The ethyl acetate was then washed with brine, dried, and concentrated to afford 0.697 g (87%) of 4-chloro-6-ethoxy-7-isopropoxy-quinazoline, mp 104-106° C. ¹H NMR (CDCl₃): δ8.81 (s, 1 H); 7.35 (s, 1 H); 7.29(s, 1 H); 4.77 (m, 1 H); 4.23 (q 2 H); 1.54 (t, 3 H); 1.47 (d, 6 H). MS (API): m/z 267 ,269 (M+H⁺, chlorine isotope pattern).

### Preparation 2a

3-(2-Methyl-thiazol-4-yl)-phenylamine. 2-Methyl-4-(3-nitrophenyl)-thiazole (9.19 g) was added in three portions over 10 minutes to a refluxing solution of tin(II) chloride dihydrate (31.05 g, 0.137 mol) in ethanol (100 mL) and 37% aqueous hydrochloric acid (50 mL). Heating was continued for 20 minutes longer, then the solution was cooled and poured into a cold solution of 120 g of potassium hydroxide in 1 L of water with stirring. Stirring with ice cooling was continued for 15 minutes, then the precipitate was filtered, washed with water and dried to afford 6.87 g (86%) of 3-(2-methyl-thiazol-4-yl)-phenylamine, mp 102-103° C. ¹H NMR (CDCl₃): δ7.25 (m, 2 H); 7.20 (m, 2 H); 6.66 (d, 1 H); 2.75 (s, 3 H). MS (API): m/z =191 (M+H⁺). Analysis (C₁₀H₁₀N₂S): Calculated C 63.13, H 5.30, N 14.72; Found: C 63.38, H 5.31, N 14.84.

### Preparation 2b

4-(3-Aminophenyl)-thiazole-2-carboxylic acid amide. A solution of tin(II) chloride dihydrate (1.331 g, 5.9 mmol) in ethanol (10 mL) and 37% aqueous hydrochloric acid (1.8 mL) was heated to reflux and treated with 4-(3-nitrophenyl)-thiazole-2-carboxylic acid ethyl ester (0.500 g, 1.8 mmol). Heating was continued for 40 minutes, after which the reaction mixture was cooled and poured into a solution of 4.0 g of potassium hydroxide in 50 mL of water with ice cooling. The precipitated product was extracted with ethyl ether. The ether extract was washed with water, brine, dried and concentrated to afford 0.266 g (60%) of 4-(3-aminophenyl)-thiazole-2-carboxylic acid ethyl ester as a viscous syrup. ¹H NMR (CDCl₃): δ7.68 (s, 1 H); 7.35 (m, 1 H); 7.21 (m, 2 H); 6.69 (d of t, 1 H); 4.48 (q, 2 H); 1.43 (t, 3 H). MS (API): m/z 249 (M+H⁺). This was dissolved in 15 mL of ethanol and the solution was saturated with dry ammonia gas with cooling in ice. The mixture was tightly stoppered and stirred at room temperature overnight. The reaction mixture was concentrated to dryness to afford 0.223 g (92%) of 4-(3-aminophenyl)-thiazole-2-carboxylic acid amide, mp 193-194° C. ¹H NMR (dmso-d₆): δ8.16 (s, 1 H); 8.13 (br, 1 H); 7.93 (br, 1 H); 7.22 (m, 2 H); 7.14 (t, 1 H); 6.59 (d of t, 1 H); 5.18 (br, 2 H). MS (API): m/z 220 (M+H⁺). Analysis (C₁₀OH₉N₃OS): Calculated: C 54.78, H 4.14, N 19.16; Found: C 54.95, H 4.09, N 19.20.

### Preparation 2c

3-Pyridin-3-yl-phenylamine. A solution of 0.744 g (3.3 mmol) of tin(II) chloride dihydrate in 6 mL of ethanol and 1 mL of 37% aqueous hydrochloric acid was heated to reflux and treated with 0.197 g (0.98 mmol) of 3-(3-nitrophenyl)-pyridine (J. Chem. Soc. 1940, 349). Heating was continued for 30 minutes, after which the reaction mixture was cooled and poured into a solution of 5.0 g of potassium hydroxide in 30 mL of water with ice cooling. The mixture was extracted with ethyl ether. The ether was washed with water, brine, dried, and concentrated to afford 0.141 g (84%) of the title product as a viscous oil that crystallized on standing, mp 70-72° C. ¹H NMR (CDCl₃): δ8.80 (d, 1 H); 8.54 (m, 1 H); 7.85 (d of t, 1 H); 7.34 (m, 1 H); 7.22 (m, 1 H); 6.94 (d, 1 H); 6.86 (s, 1 H); 6.70 (d of d, 1 H); 3.13 (br, 2 H). MS (API): m/z 171 (M+H⁺).

### Preparation 2d

2-(3-Aminophenyl)-thiazole-4-carboxylic acid amide. Tin(li) chloride dihydrate (14.89 g, 66 mmol) was dissolved in 20 mL of 37% aqueous hydrochloric acid with heating. Ethanol (60 mL) was added, followed by 4.98 g (20 mmol) of 2-(3-nitrophenyl)-thiazole-4-carboxylic acid amide in three portions. The mixture was heated for 25 minutes and then poured into a cold solution of 66 g of potassium hydroxide in 750 mL of water. The precipitate was filtered, washed with water and dried to afford 3.00 g (68%) of 2-(3-aminophenyl)-thiazole-4-carboxylic acid amide as a white powder, mp 173-174° C. ¹H NMR (dmso-d₆): δ8.17 (s, 1 H); 7.70 (br, 1 H); 7.59 (br, 1 H); 7.15-7.08 (m, 3 H); 6.66 (d, 1 H); 5.31 (br, 2 H). MS (API): m/z 220 (M+H⁺). Analysis (C₁₀H₉N₃0S): Calculated: C 54.78, H 4.14, N 19.16; Found: C 54.47, H 4.00, N 18.84.

### Preparation 2e

4-Fluoro-3-(2-methyl-thiazol-4-yl)-phenylamine. Tin(II) chloride dihydrate (2.22 g, 9.9 mmol) was dissolved in 3 mL of 37% aqueous hydrochloric acid with heating. Ethanol (15 mL) was added, followed by 0.72 g (3 mmol) of 4-(2-fluoro-5-nitro-phenyl)-2-methyl-thiazole. The mixture was heated for 30 minutes, then poured into a cold solution of 6.5 g of potassium hydroxide in 70 mL of water. The precipitate was filtered, washed with water and dried to afford 0.58 g (93%) of 4-fluoro-3-(2-methyl-thiazol-4-yl)-phenylamine as a white powder, mp 73-75° C. ¹H NMR (CDCl₃): δ7.58 (m, 2 H); 6.92 (m, 1 H); 6.65 (m, 1 H); 2.77 (s, 3 H). MS (API): m/z 209 (M+H⁺). Analysis (C₁₀H₉FN₂S): Calculated: C 57.67, H 4.36, N 13.45; Found: C 57.30, H 4.28, N 13.25.

### Preparation 3a

6,7-Diethoxy-3H-quinazolin-4-one. A solution of 10.13 g (40 mmol) of 2-amino-4,5-diethoxybenzoic acid ethyl ester and 6.14 g of powdered ammonium carbonate (64 mmol) in 40 mL of formamide was heated to 170° C. over 1 hour and then kept at 170° C. for 3 hours. The mixture was cooled and poured into 120 mL of water. The precipitate that formed was filtered, washed with water and dried to afford 8.45 g (90%) of 6,7-diethoxy-3H-quinazolin-4-one as a white solid, mp 255-257° C. ¹H NMR (dmso-d₆): δ7.93 (s, 1 H); 7.38 (s, 1 H); 7.07 (s, 1 H); 4.13 (q, 2H); 4.08 (q, 2 H); 1.35 (overlapping t, 6 H). MS (API): m/z 235 (M+H⁺).

### Preparation 3b

2-Chloromethyl-6,7-diethoxy-3H-quinazolin-4-one. A solution of 4.51 g (17.8 mmol) of 2-amino-4,5-diethoxybenzoic acid ethyl ester in 75 mL of chloroacetonitrile was saturated with dry hydrogen chloride gas until the precipitate that initially formed had dissolved. The mixture was stirred at 25° C. for 16 hours before being poured into a mixture of 200 mL of water and 30 mL of 28% aqueous ammonium hydroxide. The precipitate was filtered, washed with water, and suspended in ethanol. The solid was filtered, washed with ethanol and dried to afford 4.21 g (83%) of 2-chloromethyl-6,7-diethoxy-3H-quinazolin-4-one, mp 263-265 (dec) °C. ¹H NMR (dmso-d₆): δ7.38 (s, 1 H); 7.10 (s, 1 H); 4.47 (s, 2 H); 4.13 (q, 2 H); 4.09 (q, 2 H); 1.34 (overlapping t, 6 H). MS (API): m/z 283, 284 (M+H⁺, Cl isotope pattern). Analysis (C₁₃H₁₅ClN₂0₃): Calculated: C 55.23, H 5.35, N 9.91; Found: C 55.24, H 5.29, N 10.06.

### Preparation 3c

6-Ethoxy-7-isopropoxy-3H-quinazolin-4-one. A mixture of 4.86 g (19.2 mmol) of 2-amino-5-ethoxy-4-isopropoxy-benzoic acid ethyl ester and 2.54 g (24 mmol) of formamidine acetate in 60 mL of ethanol was heated at reflux for 5 hours. The mixture was cooled and filtered. The precipitate was washed with ethanol-diethyl ether (1:1 v/v) and then with diethyl ether and dried to afford 3.75 g (78%) of 6-ethoxy-7-isopropoxy-3H-quinazolin-4-one, mp 204-205° C. ¹H NMR (dmso-d₆): δ7.92 (s, 1 H); 7.39(s, 1 H); 7.08 (s, 1 H); 4.74 (m, 1 H); 4.07 (q, 2 H); 1.33 (t, 3 H); 1.29 (d, 6 H). MS (API): m/z 249 (M+H⁺). Analysis (C1₁₃H₁₆N₂O₃): Calculated: C 62.89, H 6.50, N 11.28; Found: C 62.92, H 6.68, N 11.41.

### Preparation 4a

2-Methyl-4-(3-nitrophenyl)-thiazole. A solution of 12.20 g (50 mmol) of 3-nitrophenacyl bromide (catalog #0935, Lancaster Synthesis, Inc., PO Box 1000, Windham, N.H. 03087) in 100 mL of ethanol was heated to reflux, then treated with 6.01 g (80 mmol) of thioacetamide (catalog #5531, Lancaster Synthesis, Inc., PO Box 1000, Windham, N.H. 03087) added in four portions. Heating was continued for 90 minutes, then the mixture was allowed to cool. The precipitate that formed was filtered, washed with ethanol and dried to afford 9.19 g (83%) of 2-methyl-4-(3-nitrophenyl)-thiazole, mp 102-104° C. ¹H NMR (dmso-d₆): δ8.70 (s, 1 H); 8.35 (d, 1 H); 8.23 (s, 1 H); 8.14 (d, 1 H); 7.69 (t, 1 H); 2.71 (s, 3 H). MS (API): m/z 221 (M+H⁺).

### Preparation 4b

4-(3-Nitrophenyl)-thiazole-2-carboxylic acid ethyl ester. A solution of 3-nitrophenacyl bromide (13.34 g, 55 mmol, catalog #0935, Lancaster Synthsis, Inc., PO Box 1000, Windham, N.H. 03087) in 80 mL of ethanol was heated at reflux and treated with ethyl thiooxamate (7.28 g, 55 mmol, catalog #33,028-0, Aldrich Chemical Co., Inc., PO Box 2060, Milwaukee, Wis. 53201). Heating was continued for 40 minutes, after which the reaction mixture was allowed to cool. The precipitated product was filtered, washed with ethanol and dried to afford 12.64 g (83%) of 4-(3-nitrophenyl)-thiazole-2-carboxylic acid ethyl ester, mp 149-150° C. ¹H NMR (CDCl₃): δ8.75 (m, 1 H); 8.31 (d of t, 1 H); 8.20 (d of t, 1 H); 7.89 (s, 1 H); 7.62 (t, 1 H); 4.49 (q, 2 H); 1.46 (t, 3 H). MS (El): m/z 278 (M+). Analysis (C₁₂H₁₀N₂O₄S): Calculated: C 51.79, H 3.62, N 10.07; Found: C 51.77, H 3.47, N 10.03.

### Preparation 4c

2-(3-Nitrophenyl)-thiazole-4-carboxylic acid amide.

Step A: 2-(3-Nitrophenyl)-thiazole-4-carboxylic acid ethyl ester. Ethyl bromopyruvate (12 mL, 96 mmol, (catalog #0582, Lancaster Synthsis, Inc., PO Box 1000, Windham, N.H. 03087) and 3-nitrothiobenzamide (10.93 g, 60 mmol, J. Am. Chem. Soc. 1960, 82, 2656)) were heated under reflux in 60 mL of ethanol for 30 minutes. The mixture was cooled and the precipitate was washed with ethanol and dried to afford 8.805 g (53%) of 2-(3-nitrophenyl)-thiazole-4-carboxylic acid ethyl ester as white crystals, mp 142-145° C. ¹H NMR (CDCl₃): δ8.84 (m, 1 H); 8.39 (d, 1 H); 8.32 (d, 1 H); 8.27 (s, 1 H); 7.68 (t, 1 H); 4.48 (q, 2 H); 1.46 (t, 3 H). MS (API): m/z 279 (M+H⁺). Analysis (C₁₂H₁₀N₂O₄S): Calculated: C 51.79, H 3.62, N 10.07; Found: C 51.73, H 3.58, N 10.07.

Step B: 2-(3-Nitrophenyl)-thiazole-4-carboxylic acid amide. A mixture of 8.705 g (31 mmol) of 2-(3-nitrophenyl)-thiazole-4-carboxylic acid ethyl ester and 31 mL of 4 molar aqueous potassium hydroxide in 35 mL of tetrahydrofuran and 35 mL of methanol was heated at reflux for 90 min. The mixture was cooled and concentrated. The residue was dissolved in hot water (800 mL) and filtered, then acidified with 6 molar aqueous hydrochloric acid while still hot. Filtration and drying afforded 6.33 g (80%) of the acid as white flakes. The acid was suspended in 50 mL of tetrahydrofuran and 0.5 mL of dimethylformamide was added followed by 2.31 mL (26.5 mmol) of oxalyl chloride. After gas evolution had ceased, the clear solution was poured into a mixture of 90 mL of water and 30 mL of 28% aqueous ammonium hydroxide with vigorous stirring. After stirring for 20 minutes, the precipitate was filtered, washed with water and dried to afford 6.122 g (80% from 2-(3-nitrophenyl)-thiazole-4-carboxylic acid ethyl ester) of 2-(3-nitrophenyl)-thiazole-4-carboxylic acid amide as a pale yellow powder, mp 239-241° C. ¹H NMR (dmso-d₆): δ8.82 (s, 1 H); δ8.42 (d, 1 H); 8.36 (s, 1 H); 8.31 (d, 1 H); 8.08 (br, 1 H); 7.79 (t, 1 H); 7.67 (br, 1 H). MS (API): m/z 250 (M+H⁺). Analysis (C₁₀H₇N₃O₃S): Calculated: C 48.19, H 2.83, N 16.86.; Found: C 48.38, H 2.77, N 16.68.

### Preparation 4d

4-(2-Fluoro-5-nitro-phenyl)-2-methyl-thiazole. A solution of 1.83 g (10 mmol) of 1-(2-fluoro-5-nitro-phenyl)-ethanone (J. Med. Chem. 1990, 33, 1246) in 25 mL of acetic acid was treated with 10 mL of 48% aqueous hydrobromic acid followed by 10 mL of a 1 molar solution of bromine in acetic acid. The mixture was stirred at room temperature overnight and then was poured into cold water and extracted with diethyl ether. The extract was washed twice with water, twice with 1 molar aqueous sodium bicarbonate, brine, dried and concentrated to afford 2.33 g (89%) of 2-bromo-1-(2-fluoro-5-nitro-phenyl)-ethanone as a colorless oil which crystallized slowly on standing. ¹H NMR (CDCl₃): δ8.81 (m, 1 H); 8.46 (m, 1 H); 7.37 (t, 1 H); 4.47 (s, 2 H). MS (El): m/z 261, 263 (Br isotope pattern, M⁺). This was dissolved in 20 mL of ethanol at reflux and treated with 1.07 g (14.2 mmol) of thioacetamide (catalog #5531, Lancaster Synthesis, Inc., PO Box 1000, Windham, N.H. 03087). Heating was continued for 30 minutes, then the mixture was cooled and the precipitate was filtered, washed with ethanol and dried to afford 1.82 g (86%) of 4-(2-fluoro-5-nitro-phenyl)-2-methyl-thiazole as colorless crystals, mp 160-162° C. ¹H NMR (CDCl₃): δ9.13 (m, 1 H); 8.16 (m, 1 H); 7.70 (s, 1 H); 7.27 (t, 1 H); 2.78 (s, 3 H). MS (API): m/z 239 (M+H⁺). Analysis (C₁₀H₇FN₂O₂S): Calculated: C 50.42, H 2.96, N 11.76; Found: C 50.55, H 2.78, N 11.49.

### Preparation 5a

2-Amino-4,5-diethoxybenzoic acid ethyl ester.

Step A: 4,5-Diethoxy-2-nitrobenzoic acid ethyl ester. A solution of 50 g (0.21 mol) of 3,4-diethoxybenzoic acid ethyl ester (Monatsh. Chem. 1884, 5, 81) in 220 mL of acetic acid was stirred at room temperature while 70% aqueous nitric acid (d=1.42, 80 mL) was added dropwise. The mixture was cooled as needed with a bath of cold water to maintain the temperature below 30° C. The mixture was stirred for an additional 3 hours, then was poured into 1 L of ice water. The precipitate was extracted twice with dichloromethane. The dichloromethane extracts were combined and washed twice with water, then with 1 molar aqueous sodium bicarbonate until the washes were of pH>8. The dichloromethane extracts were dried and concentrated to afford 59 g (100%) of 4,5-diethoxy-2-nitrobenzoic acid ethyl ester as colorless crystals, mp 53-54° C. ¹H NMR (CDCl₃): δ7.42 (s, 1 H); 7.03 (s, 1 H); 4.35 (q, 2 H); 4.16 (overlapping q, 4 H); 1.48 (overlapping q, 6 H); 1.33 (t, 3 H). MS (El): m/z 283 (M⁺). Analysis (C₁₃H₁₇NO₆): Calculated: C 55.12, H 6.05, N 4.94; Found: C 55.16, H 6.02, N 4.93.

Step B: 2-Amino-4,5-diethoxybenzoic acid ethyl ester. A solution of 15.7 g (55 mmol) of 4,5-diethoxy-2-nitrobenzoic acid ethyl ester was hydrogenated at 25° C. under 50 psig of hydrogen gas in ethanol (350 mL) over 1.00 g of 10% palladium on carbon until the uptake of hydrogen ceased. The mixture was then filtered and concentrated to afford 13.8 g (100%) of 2-amino-4,5-diethoxybenzoic acid ethyl ester, mp 69-71° C. ¹H NMR (CDCl₃): δ7.34 (s, 1 H); 6.12 (s, 1 H); 4.28 (q, 2 H); 3.98 (overlapping q, 4 H); 1.43 (t, 3 H); 1.34 (overlapping t, 6 H). MS (API): m/z 254 (M+H⁺). Analysis (C₁₃H₁₉NO₄): Calculated: C 61.64, H 7.56, N 5.53; Found: C 61.51, H 7.65 N 5.46.

### Preparation 5b

2-Amino-5-ethoxy-4-isopropoxy-benzoic acid ethyl ester.

Step A: 3-Ethoxy-4-hydroxybenzaldehyde (“ethyl vanillin”, (catalog #3476, Lancaster Synthesis, Inc., PO Box 1000, Windham, N.H. 03087)) was converted to 3-ethoxy-4-isopropoxybenzoic acid by alkylation with 2-bromopropane followed by oxidation with potassium permanganate as described in the literature (Appl. Microbiol. 1974, 27, 360).

Step B: 3-Ethoxy-4-isopropoxybenzoic acid (5.82 g, 26 mmol) was heated under reflux in 100 mL of ethanol containing 2.2 mL of 98% sulfuric acid for 16 hours. The mixture was cooled, concentrated, and the residue dissolved in ethyl acetate. The ethyl acetate was washed with water, 1 molar aqueous sodium bicarbonate, dried and concentrated to afford 5.80 g (94%) of 3-ethoxy-4-isopropoxybenzoic acid ethyl ester. This was subjected to nitration and hydrogenation in a manner analogous to that described for the preparation of 2-amino-4,5-diethoxybenzoic acid ethyl ester to afford 2-amino-5-ethoxy-4-isopropoxy-benzoic acid ethyl ester, mp. 91-93° C. ¹H NMR (CDCl₃): δ7.39 (s, 1 H); 6.37 (s, 1 H); 4.56 (m, 1 H); 4.30 (q, 2 H); 3.98 (q, 2 H); 1.37 (m, 12 H). MS (API): m/z 268 (M+H⁺). Analysis (C₁₄H₂₁NO₄): Calculated: C 62.90, H 7.92, N 5.24; Found: C 62.89, H 8.13, N 5.27.

1. A compound of the formula 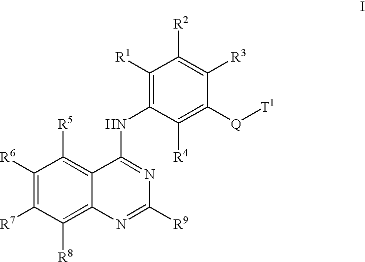
a prodrug thereof or a pharmaceutically acceptable salt of said compound or said prodrug, wherein:
Q is pyrrolyl, pyrazolyl, oxazolyl, thiazolyl, isoxazolyl, isothiazolyl, oxadiazolyl, thiadiazolyl, triazolyl, furyl, quinolyl, imidazolyl, pyridyl or pyrimidyl;
T¹ is hydrogen, methyl, ethyl, ORR¹⁰, SRR¹⁰, cyano, cyclopropyl, cyclobutyl, NH₂, NHRR¹⁰,
N(R¹⁰)₂, NHNH₂, CHRR¹⁰OH, CH₂₀RR¹⁰, COCH₃ or CON(R¹⁰)₂;
R¹, R², R³ and R⁴ are each independently hydrogen, halo, trifluoromethyl, (C₁-C₄)alkyl or (C₁-C₄)alkoxy;
R⁵and R⁸ are each independently hydrogen, fluoro, chloro, hydroxy or methyl;
R⁶ and R⁷ are each independently (C₁-C₄)alkyl or (C₁-C₄)alkoxy;
R⁹ is hydrogen, cyclopropyl, cyclobutyl, (C₁-C₄)alkyl or (CH₂)ₘ—Y;
R¹⁰, for each occurrence, is independently hydrogen, methyl or ethyl;
m is 1 , 2, 3 or 4;
Y is fluoro, chloro, bromo, hydroxy, N(R¹¹)₂, N-methylpiperazin-1-yl, thiazolidin-3-yl, thiomorpholin-4-yl, piperidin-1-yl, pyrrolidin-1-yl, morpholin-4-yl, imidazol-1-yl, (C₁-C₄)alkoxy, SR¹¹, SOR¹¹, SO₂R¹¹, CO₂H, CO₂(C₁-C₄)alkyl or CON(R¹¹)₂; and
R¹¹, for each occurrence, is independently hydrogen or (C₁-C₄)alkyl.

2. A compound of claim 1, a prodrug thereof or a pharmaceutically acceptable salt of said compound or said prodrug, wherein:
Q is oxazolyl, thiazolyl, isoxazolyl, pyridyl or pyrimidyl;
T¹ is hydrogen, methyl, OR¹⁰, CHR¹⁰OH or CON(R¹⁰)₂;
R¹, R², R³ and R⁴ are each independently hydrogen or fluoro, provided that only one of R¹, R², R³ or R⁴ is fluoro;
R⁵ and R⁸ are each hydrogen;
R⁶ is ethoxy;
R⁷ is ethoxy, n-propyl or isopropyl;
R⁹ is hydrogen or (CH₂)ₘ—Y;
Y is chloro, N(R¹¹)₂, piperidin-1-yl, pyrrolidin-1-yl, morpholin-4-yl, imidazol-1-yl, (C₁-C₄)alkoxy or SO₂R¹¹; and
R″ is methyl.

3. A compound selected from (6,7-diethoxy-quinazolin-4-yl)-(3-[1,2,4]oxadiazol-3-yl-phenyl)-amine; (6,7-diethoxy-quinazolin-4-yl)-(3-oxazol-4-yl-phenyl)-amine; (6,7-diethoxy-quinazolin-4-yl)-[3-(2-methyl-thiazol-4-yl)-phenyl]-amine; [3-(2-amino-thiazol-4-yl)-phenyl]-(6,7-diethoxy-quinazolin-4-yl)-amine; (6,7-diethoxy-quinazolin-4-yl)-(3-thiazol-4-yl-phenyl)-amine; (6,7-diethoxy-quinazolin-4-yl)-[3-(2-methyl-oxazol-5-yl)-phenyl]-amine; (6,7-diethoxy-quinazolin-4-yl)-(3-pyrimidin-5-yl-phenyl)-amine; (6,7-diethoxy-quinazolin-4-yl)-[3-(2H-pyrazol-3-yl)-phenyl]-amine; (6,7-diethoxy-quinazolin-4-yl)-(3-thiazol-5-yl-phenyl)-amine; (6,7-diethoxy-quinazolin-4-yl)-(3-thiazol-2-yl-phenyl)-amine; (6,7-dimethoxy-quinazolin-4-yl)-(3-thiazol-2-yl-phenyl)-amine; (6,7-diethoxy-quinazolin-4-yl)-(3-furan-2-yl-phenyl)-amine; (6,7-dimethoxy-quinazolin-4-yl)-(3-furan-2-yl-phenyl)-amine; (6,7-diethoxy-quinazolin-4-yl)-(3-oxazol-2-yl-phenyl)-amine; (6,7-diethoxy-quinazolin-4-yl)-[3-(2H-[1,2,4]triazol-3-yl)-phenyl]-amine; (6,7-diethoxy-quinazolin-4-yl)-[3-(2-methyl-2H-[1,2,4]triazol-3-yl)-phenyl]-amine; (6,7-dimethoxy-quinazolin-4-yl)-[3-(2-methyl-thiazol-4-yl)-phenyl]-amine; (6,7-diethoxy-quinazolin-4-yl)-[3-(2-dimethylamino-thiazol-4-yl)-phenyl]-amine; (6,7-diethoxy-quinazolin-4-yl)-[3-(2-ethoxy-thiazol-4-yl)-phenyl]-amine; (6,7-diethoxy-quinazolin-4-yl)-[3-(2-methoxy-thiazol-4-yl)-phenyl]-amine; 4-[3-(6,7-diethoxy-quinazolin-4-ylamino)-phenyl]-thiazole-2-carboxylic acid amide; (6,7-diethoxy-quinazolin-4-yl)-[3-(2-hydrazino-thiazol-4-yl)-phenyl]-amine; (6,7-diethoxy-quinazolin-4-yl)-[3-(2-methoxymethyl-thiazol-4-yl)-phenyl]-amine; (6,7-diethoxy-2-ethyl-quinazolin-4-yl)-[3-(2-methyl-thiazol-4-yl)-phenyl]-amine; (6,7-diethoxy-2-methyl-quinazolin-4-yl)-[3-(2-methyl-thiazol-4-yl)-phenyl]-amine; (6,7-diethoxy-2-isopropyl-quinazolin-4-yl)-[3-(2-methyl-thiazol-4-yl)-phenyl]-amine; (6,7-diethoxy-quinazolin-4-yl)-[3-(2-methylamino-thiazol-4-yl)-phenyl]-amine; [3-(2-cyclopropyl-thiazol-4-yl)-phenyl]-(6,7-diethoxy-quinazolin-4-yl)-amine; (6,7-diethoxy-2-propyl-quinazolin-4-yl)-[3-(2-methyl-thiazol-4-yl)-phenyl]-amine; (6,7-diethoxy-2-methoxymethyl-quinazolin-4-yl)-[3-(2-methyl-thiazol-4-yl)-phenyl]-amine; (2-cyclopropyl-6,7-diethoxy-quinazolin-4-yl)-[3-(2-methyl-thiazol-4-yl)-phenyl]-amine; (6,7-diethoxy-2-methylsulfanylmethyl-quinazolin-4-yl)-[3-(2-methyl-thiazol-4-yl)-phenyl]-amine; (6,7-diethoxy-2-methanesulfonylmethyl-quinazolin-4-yl)-[3-(2-methyl-thiazol-4-yl)-phenyl]-amine; (6,7-diethoxy-2-isobutyl-quinazolin-4-yl)-[3-(2-methyl-thiazol-4-yl)-phenyl]-amine; (6-ethoxy-7-methoxy-quinazolin-4-yl)-[3-(2-methyl-thiazol-4-yl)-phenyl]-amine; (7-ethoxy-6-methoxy-quinazolin-4-yl)-[3-(2-methyl-thiazol-4-yl)-phenyl]-amine; [2-(2-chloro-ethyl)-6,7-diethoxy-quinazolin-4-yl]-[3-(2-methyl-thiazol-4-yl)-phenyl]-amine; {6,7-diethoxy-4-[3-(2-methyl-thiazol-4-yl)-phenylamino]-quinazolin-2-yl}-acetic acid; (2-chloromethyl-6,7-diethoxy-quinazolin-4-yl)-[3-(2-methyl-thiazol-4-yl)-phenyl]-amine; {6,7-diethoxy-4-[3-(2-methyl-thiazol-4-yl)-phenylamino]-quinazolin-2-yl}-acetic acid ethyl ester; (2-butyl-6,7-diethoxy-quinazolin-4-yl)-[3-(2-methyl-thiazol-4-yl)-phenyl]-amine; {6,7-diethoxy-4-[3-(2-methyl-thiazol-4-yl)-phenylaminol-quinazolin-2-yl}-acetic acid methyl ester; (2-diethylaminomethyl-6,7-diethoxy-quinazolin-4-yl)-[3-(2-methyl-thiazol-4-yl)-phenyl]-amine; (6,7-diethoxy-2-ethylaminomethyl-quinazolin-4-yl)-[3-(2-methyl-thiazol-4-yl)-phenyl]-amine; (2-dimethylaminomethyl-6,7-diethoxy-quinazolin-4-yl)-[3-(2-methyl-thiazol-4-yl)-phenyl]-amine; (6,7-diethoxy-2-morpholin-4-ylmethyl-quinazolin-4-yl)-[3-(2-methyl-thiazol-4-yl)-phenyl]-amine; (6,7-diethoxy-2-piperidin-1-ylmethyl-quinazolin-4-yl)-[3-(2-methyl-thiazol-4-yl)-phenyl]-amine; (6,7-diethoxy-2-pyrrolidin-1-ylmethyl-quinazolin-4-yl)-[3-(2-methyl-thiazol-4-yl)-phenyl]-amine; [6,7-diethoxy-2-(4-methyl-piperazin-1-ylmethyl)-quinazolin-4-yl]-[3-(2-methyl-thiazol-4-yl)-phenyl]-amine; (6,7-diethoxy-2-methylaminomethyl-quinazolin-4-yl)-[3-(2-methyl-thiazol-4-yl)-phenyl]-amine; {6,7-diethoxy-4-[3-(2-methyl-thiazol-4-yl)-phenylamino]-quinazolin-2-yl}-acetic acid; (6,7-diethoxy-quinazolin-4-yl)-[4-fluoro-3-(2-methyl-thiazol-4-yl)-phenyl]-amine; [4-chloro-3-(2-methyl-thiazol-4-yl)-phenyl]-(6,7-diethoxy-quinazolin-4-yl)-amine; (6,7-diethoxy-quinazolin-4-yl)-[2-fluoro-5-(2-methyl-thiazol-4-yl)-phenyl]-amine; [2-chloro-5-(2-methyl-thiazol-4-yl)-phenyl]-(6,7-diethoxy-quinazolin-4-yl)-amine; (6,7-diethoxy-quinazolin-4-yl)-[2-methyl-5-(2-methyl-thiazol-4-yl)-phenyl]-amine; (6,7-diethoxy-quinazolin-4-yl)-[2-methoxy-5-(2-methyl-thiazol-4-yl)-phenyl]-amine; (6,7-diethoxy-quinazolin-4-yl)-(3-pyridin-3-yl-phenyl)-amine; (2-aminomethyl-6,7-diethoxy-quinazolin-4-yl)-[3-(2-methyl-thiazol-4-yl)-phenyl]-amine; (6,7-diethoxy-quinazolin-4-yl)-[3-(2,5-dimethyl-thiazol-4-yl)-phenyl]-amine; [2-(2-amino-ethyl)-6,7-diethoxy-quinazolin-4-yl]-[3-(2-methyl-thiazol-4-yl)-phenyl]-amine; [2-(3-chloro-propyl)-6,7-diethoxy-quinazolin-4-yl]-[3-(2-methyl-thiazol-4-yl)-phenyl]-amine; (6,7-diethoxy-2-thiazolidin-3-ylmethyl-quinazolin-4-yl)-[3-(2-methyl-thiazol-4-yl)-phenyl]-amine; (7-isopropoxy-6-methoxy-quinazolin-4-yl)-[3-(2-methyl-thiazol-4-yl)-phenyl]-amine; 4-[3-(6,7-diethoxy-2-thiazolidin-3-ylmethyl-quinazolin-4-ylamino)-phenyl]-thiazole-2-carboxylic acid amide; 4-[5-(6,7-diethoxy-2-thiazolidin-3-ylmethyl-quinazolin-4-ylamino)-2-fluoro-phenyl]-thiazole-2-carboxylic acid amide; (6,7-diethoxy-2-piperazin-1-ylmethyl-quinazolin-4-yl)-[3-(2-methyl-thiazol-4-yl)-phenyl]-amine; 2-[3-(6,7-diethoxy-quinazolin-4-ylamino)-phenyl]-thiazole-4-carboxylic acid amide; (6,7-diethoxy-quinazolin-4-yl)-[3-(4-methyl-thiazol-2-yl)-phenyl]-amine; 4-[5-(2-dimethylaminomethyl-6,7-diethoxy-quinazolin-4-ylamino)-2-fluoro-phenyl]-thiazole-2-carboxylic acid amide; 4-[3-(2-dimethylaminomethyl-6,7-diethoxy-quinazolin-4-ylamino)-phenyl]-thiazole-2-carboxylic acid amide; (2-dimethylaminomethyl-6,7-diethoxy-quinazolin-4-yl)-[4-fluoro-3-(2-methyl-thiazol-4-yl)-phenyl]-amine; 4-[3-(6,7-diethoxy-2-morpholin-4-ylmethyl-quinazolin-4-ylamino)-phenyl]-thiazole-2-carboxylic acid amide; 4-[5-(6,7-diethoxy-2-morpholin-4-ylmethyl-quinazolin-4-ylamino)-2-fluoro-phenyl]-thiazole-2-carboxylic acid amide; (6,7-diethoxy-2-morpholin-4-ylmethyl-quinazolin-4-yl)-[4-fluoro-3-(2-methyl-thiazol-4-yl)-phenyl]-amine; (7-sec-butoxy-6-methoxy-quinazolin-4-yl)-[3-(2-methyl-thiazol-4-yl)-phenyl]-amine; (6-methoxy-7-propoxy-quinazolin-4-yl)-[3-(2-methyl-thiazol-4-yl)-phenyl]-amine; 2-[3-(6,7-diethoxy-2-thiazolidin-3-ylmethyl-quinazolin-4-ylamino)-phenyl]-thiazole-4-carboxylic acid amide; (6,7-diethoxy-2-thiazolidin-3-ylmethyl-quinazolin-4-yl)-[3-(4-methyl-thiazol-2-yl)-phenyl]-amine; (6,7-diethoxy-2-thiazolidin-3-ylmethyl-quinazolin-4-yl)-[4-fluoro-3-(2-methyl-thiazol-4-yl)-phenyl]-amine; (6,7-diethoxy-2-thiomorpholin-4-ylmethyl-quinazolin-4-yl)-[4-fluoro-3-(2-methyl-thiazol-4-yl)-phenyl]-amine; 4-[5-(6,7-diethoxy-2-thiomorpholin-4-ylmethyl-quinazolin-4-ylamino)-2-fluoro-phenyl]-thiazole-2-carboxylic acid amide; 4-[3-(6,7-diethoxy-2-thiomorpholin-4-ylmethyl-quinazolin-4-ylamino)-phenyl]-thiazole-2-carboxylic acid amide; 4-[3-(6,7-diethoxy-2-pyrazol-1-ylmethyl-quinazolin-4-ylamino)-phenyl]-thiazole-2-carboxylic acid amide; (6,7-diethoxy-2-pyrazol-1-ylmethyl-quinazolin-4-yl)-[3-(2-methyl-thiazol-4-yl)-phenyl]-amine; {4-[3-(6,7-diethoxy-quinazolin-4-ylamino)-phenyl]-thiazol-2-yl}-methanol; (6,7-diethoxy-2-imidazol-1-ylmethyl-quinazolin-4-yl)-[3-(2-methyl-thiazol-4-yl)-phenyl]-amine; (6,7-diethoxy-2-thiomorpholin-4-ylmethyl-quinazolin-4-yl)-[3-(2-methyl-thiazol-4-yl)-phenyl]-amine; (6-ethoxy-7-propoxy-quinazolin-4-yl)-[3-(2-methyl-thiazol-4-yl)-phenyl]-amine; (6-ethoxy-7-isopropoxy-quinazolin-4-yl)-[3-(2-methyl-thiazol-4-yl)-phenyl]-amine; 4-[3-(6,7-diethoxy-2-piperidin-1-ylmethyl-quinazolin-4-ylamino)-phenyl]-thiazole-2-carboxylic acid amide; 4-[5-(6,7-diethoxy-2-piperidin-1-ylmethyl-quinazolin-4-ylamino)-2-fluoro-phenyl]-thiazole-2-carboxylic acid amide; (6,7-diethoxy-2-piperidin-1-ylmethyl-quinazolin-4-yl)-[4-fluoro-3-(2-methyl-thiazol-4-yl)-phenyl]-amine; 4-[3-(6,7-diethoxy-2-pyrrolidin-1-ylmethyl-quinazolin-4-ylamino)-phenyl]-thiazole-2-carboxylic acid amide; 4-[5-(6,7-diethoxy-2-imidazol-1-ylmethyl-quinazolin-4-ylamino)-2-fluoro-phenyl]-thiazole-2-carboxylic acid amide; 4-{5-[6,7-diethoxy-2-(4-methyl-piperazin-1-ylmethyl)-quinazolin-4-ylamino]-2-fluoro-phenyl}-thiazole-2-carboxylic acid amide; (6,7-diethoxy-2-pyrrolidin-1-ylmethyl-quinazolin-4-yl)-[4-fluoro-3-(2-methyl-thiazol-4-yl)-phenyl]-amine; 4-[5-(6,7-diethoxy-2-pyrrolidin-1-ylmethyl-quinazolin-4-ylamino)-2-fluoro-phenyl]-thiazole-2-carboxylic acid amide; (6,7-diethoxy-quinazolin-4-yl)-[2-ethoxy-5-(2-methyl-thiazol-4-yl)-phenyl]-amine; (6,7-diethoxy-quinazolin-4-yl)-[2-ethyl-5-(2-methyl-thiazol-4-yl)-phenyl]-amine; (6,7-diethoxy-quinazolin-4-yl)-(3-pyridin-2-yl-phenyl)-amine; 4-[3-(6-ethoxy-7-propoxy-quinazolin-4-ylamino)-phenyl]-thiazole-2-carboxylic acid amide; 4-[3-(6-ethoxy-7-isopropoxy-quinazolin-4-ylamino)-phenyl]-thiazole-2-carboxylic acid amide; 4-[3-(7-sec-butoxy-6-methoxy-quinazolin-4-ylamino)-phenyl]-thiazole-2-carboxylic acid amide; (6-ethoxy-7-propoxy-quinazolin-4-yl)-[4-fluoro-3-(2-methyl-thiazol-4-yl)-phenyl]-amine; (6-ethoxy-7-isopropoxy-quinazolin-4-yl)-[4-fluoro-3-(2-methyl-thiazol-4-yl)-phenyl]-amine; 4-[5-(7-sec-butoxy-6-methoxy-quinazolin-4-ylamino)-2-fluoro-phenyl]-thiazole-2-carboxylic acid amide; 4-[5-(6-ethoxy-7-isopropoxy-quinazolin-4-ylamino)-2-fluoro-phenyl]-thiazole-2-carboxylic acid amide; {4-[3-(6-ethoxy-7-isopropoxy-quinazolin-4-ylamino)-phenyl]-thiazol-2-yl}-methanol; 2-[3-(6-ethoxy-7-isopropoxy-quinazolin-4-ylamino)-phenyl]-thiazole-4-carboxylic acid amide; 4-[3-(7-sec-butoxy-6-ethoxy-quinazolin-4-ylamino)-phenyl]-thiazole-2-carboxylic acid amide; {4-[3-(7-sec-butoxy-6-ethoxy-quinazolin-4-ylamino)-phenyl]-thiazol-2-yl}-methanol; 2-[3-(7-sec-butoxy-6-ethoxy-quinazolin-4-ylamino)-phenyl]-thiazole-4-carboxylic acid amide; {4-[3-(2-dimethylaminomethyl-6,7-diethoxy-quinazolin-4-ylamino)-phenyl]-thiazol-2-yl}-methanol; [2-(2-dimethylamino-ethyl)-6,7-diethoxy-quinazolin-4-yl]-[3-(2-methyl-thiazol-4-yl)-phenyl]-amine; 4-[3-(6,7-diethoxy-2-methoxymethyl-quinazolin-4-ylamino)-phenyl]-thiazole-2-carboxylic acid amide; {4-[3-(6,7-diethoxy-2-methoxymethyl-quinazolin-4-ylamino)-phenyl]-thiazol-2-yl}-methanol; {4-[3-(6,7-diethoxy-2-morpholin-4-ylmethyl-quinazolin-4-ylamino)-phenyl]-thiazol-2-yl}-methanol; 2-[3-(6,7-diethoxy-2-morpholin-4-ylmethyl-quinazolin-4-ylamino)-phenyl]-thiazole-5-carboxylic acid amide; (4-{3-[6,7-diethoxy-2-(4-methyl-piperazin-1-ylmethyl)-quinazolin-4-ylamino]-phenyl}-thiazol-2-yl)-methanol; 2-{3-[6,7-diethoxy-2-(4-methyl-piperazin-1-ylmethyl)-quinazolin-4-ylamino]-phenyl}-thiazole-5-carboxylic acid amide; 2-[3-(6,7-diethoxy-2-imidazol-1-ylmethyl-quinazolin-4-ylamino)-phenyl]-thiazole-4-carboxylic acid amide; {4-[3-(6,7-diethoxy-2-imidazol-1-ylmethyl-quinazolin-4-ylamino)-phenyl]-thiazol-2-yl}-methanol; (6,7-diethoxy-2-methoxymethyl-quinazolin-4-yl)-(3-pyridin-2-yi-phenyl)-amine; (6,7-diethoxy-quinazolin-4-yl)-(3-quinolin-3-yl-phenyl)-amine; [6,7-diethoxy-2-(2-morpholin-4-yl-ethyl)-quinazolin-4-yl]-[3-(2-methyl-thiazol-4-yl)-phenyl]-amine; 4-{3-[6,7-diethoxy-2-(2-morpholin-4-yl-ethyl)-quinazolin-4-ylamino]-phenyl}-thiazole-2-carboxylic acid amide; (6,7-diethoxy-quinazolin-4-yl)-[3-(2-methyl-oxazol-4-yl)-phenyl]-amine; 4-[3-(6,7-diethoxy-2-imidazol-1-ylmethyl-quinazolin-4-ylamino)-phenyl]-thiazole-2-carboxylic acid amide; (6,7-diethoxy-quinazolin-4-yl)-(3-pyrimidin-4-yl-phenyl)-amine; 4-[3-(6-ethoxy-7-isopropoxy-2-methoxymethyl-quinazolin-4-ylamino)-phenyl]-thiazole-2-carboxylic acid amide; {4-[3-(6-ethoxy-7-isopropoxy-2-methoxymethyl-quinazolin-4-ylamino)-phenyl]-thiazol-2-yl}-methanol; (2-chloromethyl-6-ethoxy-7-isopropoxy-quinazolin-4-yl)-[3-(2-methyl-thiazol-4-yl)-phenyl]-amine; (6-ethoxy-7-isopropoxy-2-morpholin-4-ylmethyl-quinazolin-4-yl)-[3-(2-methyl-thiazol-4-yl)-phenyl]-amine; {4-[3-(6-ethoxy-7-isopropoxy-2-morpholin-4-ylmethyl-quinazolin-4-ylamino)-phenyl]-thiazol-2-yl}-methanol; 2-[3-(6-ethoxy-7-isopropoxy-2-morpholin-4-ylmethyl-quinazolin-4-ylamino)-phenyl]-thiazole-4-carboxylic acid amide; 2-[3-(6,7-diethoxy-quinazolin-4-ylamino)-phenyl]-thiazole-4-carbonitrile; (6-ethoxy-7-isopropoxy-quinazolin-4-yl)-(3-thiazol-5-yl-phenyl)-amine; 2-[3-(6-ethoxy-7-isopropoxy-quinazolin-4-ylamino)-phenyl]-thiazole-4-carbonitrile; (6-ethoxy-2-imidazol-1-ylmethyl-7-isopropoxy-quinazolin-4-yl)-[3-(2-methyl-thiazol-4-yl)-phenyl]-amine; 4-[3-(6-ethoxy-2-imidazol-1-ylmethyl-7-isopropoxy-quinazolin-4-ylamino)-phenyl]-thiazole-2-carboxylic acid amide; {4-[3-(6-ethoxy-2-imidazol-1-ylmethyl-7-isopropoxy-quinazolin-4-ylamino)-phenyl]-thiazol-2-yl}-methanol; 2-[3-(6-ethoxy-2-imidazol-1-ylmethyl-7-isopropoxy-quinazolin-4-ylamino)-phenyl]-thiazole-4-carboxylic acid amide; 4-[3-(2-dimethylaminomethyl-6-ethoxy-7-isopropoxy-quinazolin-4-ylamino)-phenyl]-thiazole-2-carboxylic acid amide; {4-[3-(2-dimethylaminomethyl-6-ethoxy-7-isopropoxy-quinazolin-4-ylamino)-phenyl]-thiazol-2-yl}-methanol; and {4-[3-(2-chloromethyl-6-ethoxy-7-isopropoxy-quinazolin-4-ylamino)-phenyl]-thiazol-2-yl}-methanol.

4. A compound of claim 3 selected from {4-[3-(6,7-diethoxy-2-imidazol-1-ylmethyl-quinazolin-4-ylamino)-phenyl]-thiazol-2-yl}-methanol; [2-(2-amino-ethyl)-6,7-diethoxy-quinazolin-4-yl]-[3-(2-methyl-thiazol-4-yl)-phenyl]-amine; (6,7-diethoxy-quinazolin-4-yl)-(3-thiazol-2-yl-phenyl)-amine; (6,7-diethoxy-quinazolin-4-yl)-(3-thiazol-4-yl-phenyl)-amine; (6,7-diethoxy-quinazolin-4-yl)-(3-pyridin-2-yl-phenyl)-amine; (6-ethoxy-7-isopropoxy-quinazolin-4-yl)-[4-fluoro-3-(2-methyl-thiazol-4-yl)-phenyl]-amine; (6-ethoxy-7-isopropoxy-quinazolin-4-yl)-[3-(2-methyl-thiazol-4-yl)-phenyl]-amine; {4-[3-(6-ethoxy-7-isopropoxy-quinazolin-4-ylamino)-phenyl]-thiazol-2-yl}-methanol; (2-chloromethyl-6-ethoxy-7-isopropoxy-quinazolin-4-yl)-[3-(2-methyl-thiazol-4-yl)-phenyl]-amine; (6,7-diethoxy-quinazolin-4-yl)-[2-fluoro-5-(2-methyl-thiazol-4-yl)-phenyl]-amine; (6,7-diethoxy-quinazolin-4-yl)-[3-(2-methyl-oxazol-4-yl)-phenyl]-amine; [3-(6-ethoxy-7-isopropoxy-2-methoxymethyl-quinazolin-4-ylamino)-phenyl]-thiazole-2-carboxylic acid amide; {4-[3-(7-sec-butoxy-6-ethoxy-quinazolin-4-ylamino)-phenyl]-thiazol-2-yl}-methanol; 2-[3-(6,7-diethoxy-2-imidazol-1-ylmethyl-quinazolin-4-ylamino)-phenyl]-thiazole-4-carboxylic acid amide; (6,7-diethoxy-quinazolin-4-yl)-[4-fluoro-3-(2-methyl-thiazol-4-yl)-phenyl]-amine; 4-[3-(6-ethoxy-7-isopropoxy-quinazolin-4-ylamino)-phenyl]-thiazole-2-carboxylic acid amide; (6,7-diethoxy-quinazolin-4-yl)-[3-(2-methyl-thiazol-4-yl)-phenyl]-amine; (6,7-diethoxy-quinazolin-4-yl)-[3-(2H-[1,2,4]triazol-3-yl)-phenyl]-amine; (6,7-diethoxy-quinazolin-4-yl)-[3-(4-methyl-thiazol-2-yl)-phenyl]-amine; 2-[3-(6,7-diethoxy-quinazolin-4-ylamino)-phenyl]-thiazole-4-carbonitrile; 4-[5-(6-ethoxy-7-isopropoxy-quinazolin-4-ylamino)-2-fluoro-phenyl]-thiazole-2-carboxylic acid amide; 4-[3-(6-ethoxy-2-imidazol-1-ylmethyl-7-isopropoxy-quinazolin-4-ylamino)-phenyl]-thiazole-2-carboxylic acid amide; (6,7-diethoxy-quinazolin-4-yl)-[3-(2H-pyrazol-3-yl)-phenyl]-amine; (6,7-diethoxy-2-imidazol-1-ylmethyl-quinazolin-4-yl)-[3-(2-methyl-thiazol-4-yl)-phenyl]-amine; (6,7-diethoxy-quinazolin-4-yl)-(3-oxazol-2-yl-phenyl)-amine; (6,7-diethoxy-quinazolin-4-yl)-[3-(2-methoxy-thiazol-4-yl)-phenyl]-amine; (6,7-diethoxy-quinazolin-4-yl)-[3-(2-methylamino-thiazol-4-yl)-phenyl]-amine; 2-[3-(6,7-diethoxy-quinazolin-4-ylamino)-phenyl]-thiazole-4-carboxylic acid amide; {4-[3-(6,7-diethoxy-quinazolin-4-ylamino)-phenyl]-thiazol-2-yl}-methanol; [3-(2-cyclopropyl-thiazol-4-yl)-phenyl]-(6,7-diethoxy-quinazolin-4-yl)-amine; (6,7-diethoxy-2-methylsulfanylmethyl-quinazolin-4-yl)-[3-(2-methyl-thiazol-4-yl)-phenyl]-amine; (2-chloromethyl-6,7-diethoxy-quinazolin-4-yl)-[3-(2-methyl-thiazol-4-yl)-phenyl]-amine; 4-[3-(6,7-diethoxy-quinazolin-4-ylamino)-phenyl]-thiazole-2-carboxylic acid amide; {6,7-diethoxy-4-[3-(2-methyl-thiazol-4-yl)-phenylamino]-quinazolin-2-yl}-acetic acid; (6,7-diethoxy-quinazolin-4-yl)-(3-thiazol-5-yl-phenyl)-amine; (6,7-diethoxy-quinazolin-4-yl)-[2-methoxy-5-(2-methyl-thiazol-4-yl)-phenyl]-amine; (6-ethoxy-7-propoxy-quinazolin-4-yl)-[3-(2-methyl-thiazol-4-yl)-phenyl]-amine; (6,7-diethoxy-2-methyl-quinazolin-4-yl)-[3-(2-methyl-thiazol-4-yl)-phenyl]-amine; (6-ethoxy-7-propoxy-quinazolin-4-yl)-[4-fluoro-3-(2-methyl-thiazol-4-yl)-phenyl]-amine; (6-ethoxy-7-isopropoxy-quinazolin-4-yl)-(3-thiazol-5-yl-phenyl)-amine; [2-(2-chloro-ethyl)-6,7-diethoxy-quinazolin-4-yl]-[3-(2-methyl-thiazol-4-yl)-phenyl]-amine; {4-[3-(6,7-diethoxy-2-methoxymethyl-quinazolin-4-ylamino)-phenyl]-thiazol-2-yl}-methanol; 4-[3-(6-ethoxy-7-propoxy-quinazolin-4-ylamino)-phenyl]-thiazole-2-carboxylic acid amide; (6,7-diethoxy-quinazolin-4-yl)-[2-methyl-5-(2-methyl-thiazol-4-yl)-phenyl]-amine; (6-ethoxy-7-methoxy-quinazolin-4-yl)-[3-(2-methyl-thiazol-4-yl)-phenyl]-amine; 4-[3-(7-sec-butoxy-6-ethoxy-quinazolin-4-ylamino)-phenyl]-thiazole-2-carboxylic acid amide; (6,7-diethoxy-quinazolin-4-yl)-(3-pyrimidin-5-yl-phenyl)-amine; (7-sec-butoxy-6-methoxy-quinazolin-4-yi)-[3-(2-methyl-thiazol-4-yl)-phenyl]-amine; 4-[5-(6,7-diethoxy-2-imidazol-1-ylmethyl-quinazolin-4-ylamino)-2-fluoro-phenyl]-thiazole-2-carboxylic acid amide; 4-[3-(6,7-diethoxy-2-imidazol-1-ylmethyl-quinazolin-4-ylamino)-phenyl]-thiazole-2-carboxylic acid amide; 2-[3-(7-sec-butoxy-6-ethoxy-quinazolin-4-ylamino)-phenyl]-thiazole-4-carboxylic acid amide; (6,7-diethoxy-quinazolin-4-yl)-(3-furan-2-yl-phenyl)-amine; (6,7-diethoxy-quinazolin-4-yl)-(3-[1,2,4]oxadiazol-3-yl-phenyl)-amine; (6,7-diethoxy-2-propyl-quinazolin-4-yl)-[3-(2-methyl-thiazol-4-yl)-phenyl]-amine; (6,7-diethoxy-quinazolin-4-yl)-(3-pyridin-3-yl-phenyl)-amine; (6,7-diethoxy-2-methoxymethyl-quinazolin-4-yl)-[3-(2-methyl-thiazol-4-yl)-phenyl]-amine; (2-aminomethyl-6,7-diethoxy-quinazolin-4-yl)-[3-(2-methyl-thiazol-4-yl)-phenyl]-amine; and {4-[3-(6-ethoxy-2-imidazol-1-ylmethyl-7-isopropoxy-quinazolin-4-ylamino)-phenyl]-thiazol-2-yl}-methanol.

5. A pharmaceutical composition comprising a therapeutically effective amount of a compound of claim 1, a prodrug thereof or a pharmaceutically acceptable salt of said compound or said prodrug.

6. A pharmaceutical composition of claim 5 additionally comprising a pharmaceutically acceptable carrier, vehicle or diluent.

7. A method of treating diabetes, diabetic complications or cancer in a mammal comprising administering to said mammal a pharmaceutical composition of claim 6.

8. A method of treating diabetes, diabetic complications or cancer in a mammal comprising administering to said mammal a compound of claim 1, a prodrug thereof or a pharmaceutically acceptable salt of said compound or said prodrug.

9. A method of claim 7 wherein Type I or Type II diabetes is treated.

10. A method of claim 8 wherein Type I or Type II diabetes is treated.

11. A method of claim 7 wherein one or more diabetic complications are treated.

12. A method of claim 8 wherein one or more diabetic complications are treated.

13. A method of claim 7 wherein cancer is treated.

14. A method of claim 8 wherein cancer is treated.
